(19) United States
(12) Patent Application Publication
(10) Pub. No.: US 2012/0028358 A1
(43) Pub. Date: Feb. 2, 2012

(54) METHOD FOR INCREASING RETROVIRAL INFECTIVITY

(75) Inventors: Victor Solodushko, Mobile, AL (US); Brian Fouty, Spanish Fort, AL (US)

(73) Assignee: UNIVERSITY OF SOUTH ALABAMA, Mobile, AL (US)

(21) Appl. No.: 13/170,836

(22) Filed: Jun. 28, 2011

Related U.S. Application Data
(60) Provisional application No. 61/359,223, filed on Jun. 28, 2010.

(51) Int. Cl. C12N 15/867; C12N 5/071

(52) U.S. Cl. 435/456; 435/375

(57) ABSTRACT

The present invention is directed to methods for enhanced retroviral delivery of a nucleic acid to a target cell in vitro, ex vivo or in vivo, which involves increasing infectivity of a retrovirus carrying a transgene of interest to a target cell. In one particular aspect the invention relates to a method for increasing retroviral infectivity of target cells comprising culturing packaging cells transfected with retroviral vector containing a transgene of interest in medium containing a glucocorticoid receptor agonist or analog or derivative thereof present in the medium in an amount effective to increase titer of propagated retrovirus; and subsequently culturing a target cell in medium comprising the propagated retrovirus from a) and a glucocorticoid receptor antagonist or analog or derivative thereof present in the medium in an amount effective to increase target cell sensitivity to infection by the propagated retrovirus.

### CROSS-REFERENCE TO RELATED APPLICATIONS

This application claims the benefit of the filing date of U.S. Provisional Patent Application No. 61/359,223 filed Jun. 28, 2010, the disclosure of which is hereby incorporated herein by reference.

### STATEMENT REGARDING FEDERALLY SPONSORED RESEARCH OR DEVELOPMENT

This invention was made with Government support, Grant No. RO1 HL70273-01 (BF). Thus, the Government may have certain rights in the invention.

### BACKGROUND OF THE INVENTION

Using adapted retroviruses for gene delivery is a modern and powerful tool in biological research as well as a promising approach for gene therapy. Specifically, use of modified retroviruses has been reported as an easy and safe tool for stable introduction of gene(s) into host cells (16, 27). In this system, the gene of interest is cloned into a retroviral vector that may also contain a selective marker such as an antibiotic resistance or reporter gene. Virus-producing cell lines, transfected with a retroviral vector containing the genes of interest, produce retroviral RNA packaged into viral particles. Retrovirus-containing conditioned medium from packaging cells may be directly used for transduction of dividing target cells (21). Several packaging cell systems that express a range of viral envelope proteins with unique host specificity and efficiency are available (13).

Modified moloney murine leukemia virus (MMLV) is among the most popular and widely used vehicle for stable gene delivery due to its ability to stably integrate its own genetic material, as well as the transgene, into host chromosomal DNA with low immunogenicity (2). Phoenix packaging cells are a second-generation retrovirus producer line for the generation of helper-free retroviruses. This line was created by placing constructs capable of producing gag-pol (necessary for viral replication and particle formation) and envelope proteins into 293T cells.

Typically, however, use of retroviral vectors is limited by the low infection rate in target cells due to the insufficient infectivity of standard retrovirus supernatants. Current solutions entail complicated methods of virus concentration. Unfortunately, low retroviral infection efficiency of target cells remains a limitation in using any retroviral system. In practice, most target cells remain uninfected even after several infection cycles. Resistance of cells to infection may be due among other reasons, to low numbers of cell surface receptors recognized by retroviruses and slow cell cycle progression. These limitations notwithstanding, retroviral vector-mediated gene transfer remains the easiest way for stable gene delivery into cells and can be used to permanently modify the host cell nuclear genome. As such, there remains a need for methods to increase viral titer and/or increase target cell sensitivity to retroviral infection, which can increase retroviral infectivity and thus improve the efficiency of existing retroviral delivery systems and broaden the spectrum of applications of this technique.

### SUMMARY OF THE INVENTION

In its broadest aspect, the present invention is directed to a method for enhancing or increasing infectivity of a retrovirus carrying a transgene of interest to a target cell. The method entails culturing a target cell in medium that comprises, in addition to propagated transformed retrovirus, a glucocorticoid receptor antagonist in an amount effective to increase infectivity of the retrovirus to the target cell (e.g., enhance integration of the retrovirus into the target cell). In some embodiments, the glucocorticoid receptor antagonist is mifepristone (RU-486), or an analog or derivative thereof. In some embodiments, the propagated retrovirus is obtained from a frozen stock of retroviral supernatant. The timing of the addition of the glucocorticoid receptor antagonist to the target cell cultures is not critical—it may be added before, at about the same time, or after the addition of the retrovirus. In some embodiments, the medium may contain another infectivity enhancing agent. Without intending to be bound by theory, the present inventors believe that the enhanced infectivity is achieved by increased integration of the retroviral nucleic acid (including the transgene) into the target cell genome.

In other embodiments, this aspect of the present invention may include a prior step of culturing packaging cells so as to increase titer of the virus propagated therein. Thus, in these embodiments, the present invention relates to a method for increasing retroviral infectivity of target cells comprising:
a) culturing packaging cells transfected with retroviral vector containing a transgene of interest in medium containing a glucocorticoid receptor agonist or analog or derivative thereof present in the medium in an amount effective to increase titer of propagated retrovirus; and
b) subsequently culturing a target cell in medium comprising the propagated retrovirus from a) and a glucocorticoid receptor antagonist or analog or derivative thereof present in the medium in an amount effective to increase target cell sensitivity to infection by the propagated retrovirus. In some embodiments, the glucocorticoid receptor agonist is dexamethasone, or an analog or derivative thereof. In some embodiments, the glucocorticoid receptor antagonist is mifepristone or an analog or derivative thereof. In some embodiments, the target cells are cultured with retrovirus-containing culture medium obtained from packaging cell cultures (referred to herein as “conditioned medium”), or with retrovirus that has been obtained from packaging cell culture medium according to conventional methods, for example by ultracentrifugation or column concentration or other conventional means of concentrating the retrovirus. Optionally, in some embodiments the packaging cells are cultured with a glucocorticoid receptor agonist prior to culture of the target cells with a glucocorticoid receptor antagonist, such that culture with glucocorticoid receptor agonist and glucocorticoid receptor antagonist occurs in a step-wise, or sequential fashion.

In various embodiments of the present invention the retrovirus is a gamma retrovirus.

In another aspect, the present invention relates to a kit which comprises components for carrying out the methods of the present invention. Such kits may include in separate containers, a glucocorticoid receptor agonist and a glucocorticoid receptor antagonist in amounts sufficient to increase retroviral titer and target cell sensitivity to retroviral infection, respectively. The kits may also include one or more additional retroviral infectivity enhancers.

### BRIEF DESCRIPTION OF THE DRAWINGS

FIG. 1: FIGS. 1A-1C illustrate that dexamethasone enhances expression of viral proteins and increases viral release into the supernatant. Phoenix cells were grown in increasing concentrations of dexamethasone for 36 hrs. 1A: Expression of the retroviral reporter protein, GFP, as measured by flow cytometry (mean fluorescence per cell, normalized to unstimulated cells). Gray line represent background signal for Phoenix cells without integrated provirus. 1B: Western blot of cell lysates probed with an anti-GFP antibody in 0 or 100 nmol-dexamethasone treated Phoenix cells. 1C: Quantitative PCR on the conditioned medium using primers for the GFP region of the viral RNA. (N) indicates Phoenix cells without the integrated pro-viral DNA. (n=4 experiments, *indicates p<0.05).

FIG. 2: FIGS. 2A and 2B are graphs showing that dexamethasone pretreatment increases both the number of infected target cells and the expression of viral proteins within infected cells. Conditioned medium from Phoenix cells grown in increasing concentration of dexamethasone was directly applied to rat PMVEC. 2A: The percentage of GFP-positive cells. 2B: The intensity of GFP expression levels in positive (infected) cells as measured by flow cytometry. Gray line represent background signal for non-infected PMVEC. (n=4 experiments, *indicates p<0.05).

FIG. 3: FIGS. 3A and 3B are graphs showing that adding dexamethasone to collected viral supernatant does not increase target cell infectivity, but does increase expression of viral proteins in infected cells. Conditioned medium from Phoenix cells grown without dexamethasone was collected and then supplemented with 10 nmol, 100 nmol or 1000 nmol of dexamethasone, before being added to rat PMVEC. 3A: The percentage of GFP-positive cells. 3B: GFP intensity within positive (infected) cells after 72 hours as measured by flow cytometry. Gray line represent background signal for non-infected PMVEC. (n=4 experiments, *indicates p<0.05).

FIG. 4: FIGS. 4A-4C present data which show that reducing steroid hormone levels through the use of charcoal-stripped FBS on Phoenix packaging cells decreased viral protein expression, viral titer, and target cell infectivity. Phoenix cells were grown in medium supplemented with either normal or charcoal-stripped FBS. 4A: GFP intensity in each condition as measured by flow cytometry. Upper figure is histogram representing the aggregate data from 4 experiments, below is a representative example of a single experiment. 4B: Quantitative PCR on the conditioned medium using primers for the GFP region of the viral RNA. 4C: Percent of infected target cells (rat PMVEC) incubated with conditioned medium under each condition. Upper figure is a histogram representing the aggregate data from 4 experiments, below is a representative example of a single experiment.

FIG. 5: FIGS. 5A-5C show data that indicates that mifepristone has no effect on expression of viral proteins or viral titer of supernatant in Phoenix (packaging) cells. Phoenix cells were incubated with increasing concentrations of mifepristone for 36 hrs. 5A: Expression of the retroviral reporter protein, GFP, as measured by flow cytometry (mean fluorescence per cell, normalized to vehicle-treated cells). Gray line represent background signal for Phoenix cells without integrated provirus. 5B: Western blot for GFP of Phoenix cell lysates treated with 0 or 2 μmol mifepristone. 5C: Quantitative PCR on the conditioned medium using primers for the GFP region of the viral RNA. (N) indicates conditioned medium from Phoenix cells without the integrated pro-viral DNA (n=4 experiments).

FIG. 6: FIGS. 6A and 6B are graphs which show that mifepristone added to the conditioned medium increases the percentage of infected target cells but does not alter the viral protein expression in infected cells. Phoenix cells were grown in the presence of increasing concentrations of mifepristone and the conditioned medium was then applied to rat PMVEC for 72 hours. 6A: Percent of GFP-positive (infected) cells. 6B: Intensity of GFP expression in positive (infected) cells as measured by flow cytometry. Gray line represent background signal for non-infected PMVEC. (n=4 experiments, *indicates p<0.05).

FIG. 7: FIGS. 7A and 7B are graphs illustrating that dexamethasone increases target cell infectivity by increasing viral titer, whereas mifepristone increases target cell infectivity independent of viral titer. Phoenix cells were incubated with vehicle (C), 2 μmol mifepristone (M), 100 nmol dexamethasone (D), or both (DM) for 36 hours. 7A: Quantitative PCR on the conditioned medium using primers for the GFP region of the viral RNA. 7B: Percent GFP-positive (infected) cells after exposure to the conditioned medium under each condition. Mifepristone and dexamethasone were added either to the packaging cells or to the medium after collection before applying to target cells. (n=4 experiments, *indicates p<0.05). (N) indicates data for conditioned medium from Phoenix cells without the integrated pro-viral DNA.

FIG. 8: FIGS. 8A-8C are graphs showing that the sequential addition of dexamethasone at the time of virus propagation and mifepristone at the time of infection resulted in the highest rate of target cell infectivity. Conditioned medium from Phoenix cells was applied to target cells (rat PMVEC) for 12 hours and the percent of GFP-positive (infected) cells determined 72 hours later by flow cytometry. 8A: Percent of infected target cells after incubation with conditioned medium in which 2 μmol of Mifepristone was added to Phoenix cells (M), 100 nmol dexamethasone was added to the Phoenix cells (D), or 100 nmol dexamethasone was first added to the Phoenix cells and then 2 μmol mifepristone was added to the collected medium before application to target cells (DM). (C) represents untreated cells. 8B: Representative flow cytometry tracing depicting percent of infected cells on the y-axis and intensity of GFP expression on the x-axis (data smoothed for graphing). 8C: Conditioned medium was collected and stored in a CO₂ incubator at 37° C. for the indicated times in the presence of mifepristone, dexamethasone, or both. Each 24 hours, target cells were incubated with this conditioned medium and the percent of infected cells determined 72 hours later. Infectivity (normalized to fresh conditioned medium with hormones) dropped about 85% per day, but was not changed by the presence of dexamethasone or mifepristone. (n=4 experiments, *p<0.05).

FIG. 9: FIGS. 9A and 9B are graphs showing that mifepristone increased the percentage of infected target cells (PMVEC) regardless of the type of gamma-retroviral backbone or viral envelope. 9A: Ecotropic gamma-retroviruses MMLV, MSCV, and FMEV and VSV-C pantropic lentiviruses pLemiR and pLVX-DsRed-Monomer-C1 were used to infect PMVEC in the presence of 1 μmol/L mifepristone or vehicle control. 9B: MMLV packaged in viral particles with different envelope proteins were used to infect PMVEC in the presence of 1 μmol/L mifepristone or vehicle control. The percentage of infected (fluorescent) cells was determined 72 hours after infection. (n=4 experiments, *indicates p<0.05).

FIG. 10: FIG. 10 is a graph showing that adding mifepristone after the initial retroviral infection also increased target cell infection efficiency. PMVEC were infected with ecotropic MMLV for 1 hour. After virus removal, cells were incubated in fresh medium for an additional 1 hour and then 1 μmol/L mifepristone or vehicle control was added. The percentage of GFP positive (infected) cells was measured 72 hours after infection. (n=4 experiments, *indicates p<0.05).

FIG. 11: FIGS. 11A and 11B are graphs which show that mifepristone does not increase infection efficiency by promoting infection of non-dividing cells. Growing (15% confluent) or growth-arrested (100% confluent) PMVEC were infected with ecotropic MMLV at the same MOI for 18 hours in the presence of 1 μmol/L mifepristone or vehicle control. The percentage of GFP positive (infected) cells was determined 72 hours after infection. (n=4 experiments, *indicates p≦0.05).

FIG. 12: FIGS. 12A-12E illustrate that mifepristone had no effect on the survival or stability of the non-integrated gamma-retrovirus in infected cells. Growth-arrested PMVEC from a confluent monolayer were trypsinized and re-seeded at 15% confluency. Cell cycle progression was monitored by PI staining using FACS. 12A: Representative data of cell cycle progression 24, 30 and 36 hours after cell re-seeding. 12B: Statistical data for the percentage of G2-M phase endothelial cells at different time points after re-seeding. PMVEC enter G2-M phase 30 hours after release from confluency-induced growth arrest. Only cells that enter G2-M phase can be infected by gamma-retroviruses. After re-seeding from confluent monolayer, cells were also incubated with an ecotropic MMLV retrovirus encoding GFP for 12 hours in the presence or absence of 1 μmol/L mifepristone immediately, 12, or 36 hours after re-seeding to lower confluency. The percentage of GFP-positive (infected) cells was measured 72 hours after infection. 12C: Representative data showing the infection efficiency of PMVEC infected with MMLV at different time periods after re-seeding in the presence or absence of mifepristone. 12D: Statistical data indicating the percentage of infected endothelial cells at different periods after re-seeding. 12E: The relative enhancing effect of mifepristone on retroviral infection at different periods after re-seeding. (n=4 experiments, *indicates p<0.05).

FIG. 13: FIGS. 13A-13C are bar graphs showing that mifepristone enhances viral integration into host DNA. PMVEC were infected with ecotropic MMLV for 1 hour in the presence of 1 μmol/L mifepristone or vehicle control. The virus was removed by washing and cells were then cultured in fresh medium supplemented with 1 μmol/L mifepristone or vehicle until analysis. (13A) Total DNA (host and viral) was extracted from target cells and the presence of viral DNA (using primers to the GFP coding sequence that is present only in the viral DNA) was determined.

Total target cell DNA was extracted and then separated in a 0.8% agarose gel. Non-integrated viral DNA migrates to about 4 Kb whereas genomic DNA migrates >50 Kb. Each fraction was extracted from the gel and analyzed for the presence of viral DNA separately. For each time point, equal amounts of DNA were examined. (13B) qPCR from the 4 Kb fragment representing the relative amounts of non-integrated viral DNA in mifepristone or vehicle treated cells. (13C) qPCR from the >50 Kb genomic DNA representing the relative amounts of integrated viral DNA in mifepristone or vehicle treated cells. qPCR results in each experimental design were normalized to the signal obtained from vehicle-treated cells 1 hour after infection. DNA was extracted and analyzed 1, 6 and 24 hours and also 3 and 7 days after initial infection. These qPCR results were confirmed using another set of primers encoding for the internal ribosome entry site of viral DNA (present in the pBMN-GFP vector). (n=4 experiments, *indicates p<0.05).

FIG. 14: FIGS. 14A and 14B are graphs showing that mifepristone increases gamma-retroviral infection efficiency through its effect on the glucocorticoid receptor. 14A: PMVEC were infected with ecotropic MMLV in the presence of increasing concentrations of the glucocorticoid receptor antagonists—mifepristone or Org 34517, or the progesterone receptor antagonist, 7453-102. The percentage of GFP positive (infected) cells was determined 72 hours after infection. 14B: Two different human cell lines (U87MG which contain no functional progesterone receptors, and T47D which contain no functional glucocorticoid receptors) were infected with amphotropic MMLV in the presence of 1 μmol/L mifepristone or vehicle control. The percentage of GFP-positive (infected) cells was determined 72 hours after initial infection. Mifepristone did not increase infection efficiency in the T47D cells. (n=4 experiments, *indicates p<0.05).

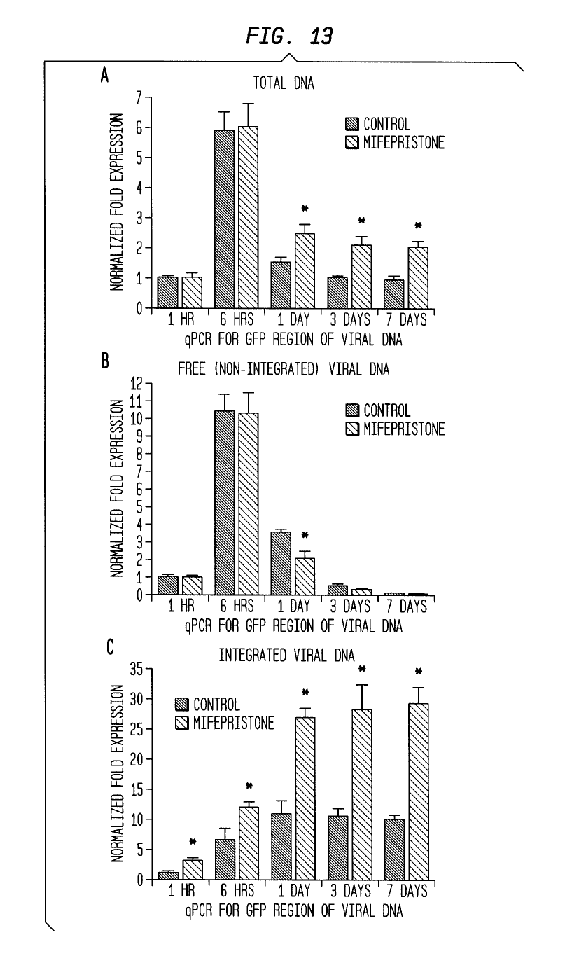

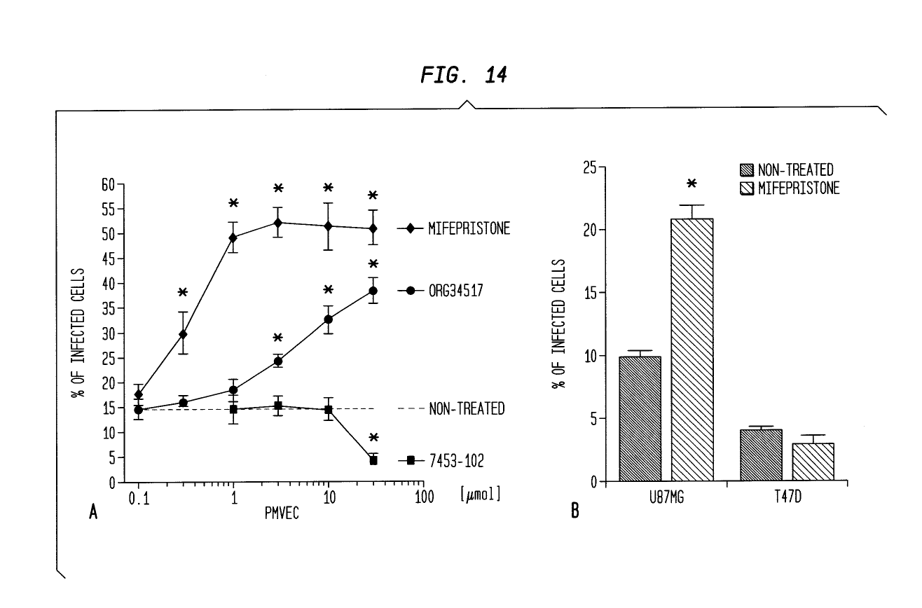

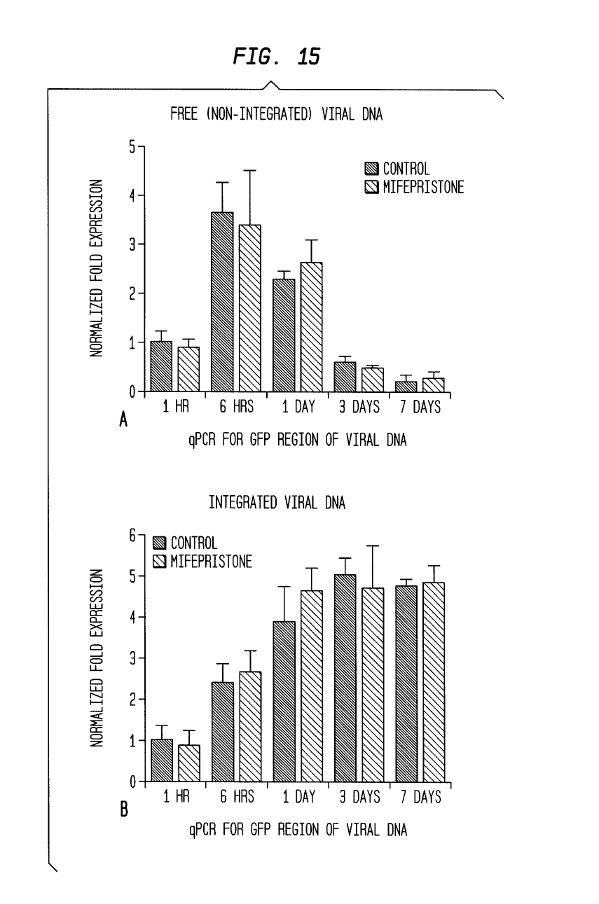

FIG. 15: FIGS. 15A and 15B are graphs showing that mifepristone does not increase viral integration into host DNA of T47D cells. T47D cells were infected with amphotropic MMLV for 1 hour in the presence of 1 μmol/L mifepristone or vehicle control (using a similar protocol as described in FIG. 13). The virus was removed by washing and cells were then cultured in fresh medium supplemented with 1 μmol/L mifepristone or vehicle until analysis. Non-integrated viral and genomic DNA were separated in and extracted from a 0.8% agarose gel at same way as was described for PMVEC and analyzed for the presence of viral DNA separately. For each time point, equal amounts of DNA were examined. 15A: qPCR from the 4 Kb fragment representing the relative amounts of non-integrated viral DNA in mifepristone or vehicle treated T47D cells. 15B: qPCR from the >50 Kb genomic DNA representing the relative amounts of integrated viral DNA in mifepristone or vehicle treated cells. qPCR results in each experimental design were normalized to the signal obtained from vehicle-treated cells 1 hour after infection. DNA was extracted and analyzed 1, 6 and 24 hours and also 3 and 7 days after initial infection. These qPCR results were confirmed using another set of primers encoding for the internal ribosome entry site of viral DNA (present in the pBMN-GFP vector). (n=4 experiments, *indicates p<0.05).

### DETAILED DESCRIPTION

While the specification concludes with the claims particularly pointing out and distinctly claiming the invention, it is believed that the present invention will be better understood from the following description.

All percentages and ratios used herein are by weight of the total composition and all measurements made are at 25° C. and normal pressure unless otherwise designated. All temperatures are in Degrees Celsius unless specified otherwise. The present invention can comprise (open-ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise.

All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value. Unless otherwise indicated, as used herein, “a” and “an” include the plural, such that, e.g., “a cell” can mean more than one cell.

Conventional techniques of cell biology, molecular biology, cell culture, virology, and the like may be used to practice the methods of the present invention. These techniques are well know to one of skill in the art and are disclosed in e.g., Sambrook, Fritsch and Maniatis eds., “Molecular Cloning, A Laboratory Manual”, 2nd Ed., Cold Spring Harbor Laboratory Press (1989); Celis J. E. “Cell Biology, A Laboratory Handbook” Academic Press, Inc. (1994); Bahnson et al., J. of Virol. Methods, 54:131-143 (1995) and Berger and Kimmel eds., “Methods in Enzymology Volume 152: Guide to Molecular Cloning Techniques”, Academic Press, Inc. (1987).

Methods of gene delivery using retroviral vectors are well known in the art. Typically, a retroviral based vector may be engineered to contain a nucleic acid of interest for retroviral delivery to a target cell. The engineered retroviral vector may then be transfected into cells in vitro which permit propagation of the virus (“packaging cells”). Transfected packaging cells are cultured in conditions and for a time sufficient to permit retroviral propagation to a desired titer. Culture medium containing the propagated retrovirus (“conditioned medium”) may then be collected and used directly to infect cultures of target cells, or may be further processed to obtain a concentrated preparation of retrovirus particles which may be used to infect target cell cultures. See for example, Kurth, R; Bannert, N (editors) (2010). Retroviruses: Molecular Biology, Genomics and Pathogenesis. Caister Academic Press. ISBN 978-1-904455-55-4; Desport, M (editors) (2010). Lentiviruses and Macrophages: Molecular and Cellular Interactions. Caister Academic Press. ISBN 978-1-904455-60-8.

Retroviral infectivity of target cells may be enhanced by culturing target cells in media comprising retrovirus and a glucocorticoid receptor antagonist such as mifepristone in an amount effective to cause an increase in target cell sensitivity to retroviral infection. Optionally, this step may be preceded by propagation of the retrovirus in a culture of packaging cells comprising a glucocorticoid receptor agonist such as dexamethosone in an amount effective to stimulate an increase in retroviral titer. As a result, the methods of the present invention provide a significant improvement in the effectiveness of retroviral delivery systems by achieving greater target cell infectivity of retroviruses than what is achieved using conventional methods of retroviral propagation and infection.

It is contemplated that the methods disclosed herein to increase retroviral infectivity may be used alone or in conjunction with and/or combined with other transfection reagents or other methods or substances that can increase retroviral infectivity. For example, in addition to the use of a glucorticoid receptor antagonist (e.g., mifepristone) alone, or the use of a glucorticoid receptor agonist (e.g., dexamethasone) in combination with mifepristone according to the present invention, additional compounds may be used to increase infectivity, for example, by increasing target cell sensitivity to infection, including, e.g., by facilitating viral integration into the target cell genome. Similarly, cultures of packaging cells may include dexamethasone alone, or in combination with one or more other compounds that may be used to increase infectivity, for example, by enhancing viral production by the transfected packaging cell line and thus increasing retroviral titer. These reagents are familiar to one of skill in the art and include, for example, polybrene and many other commercially available transfection reagents.

It is further contemplated herein that glucocorticoid receptor agonists and antagonists may be added to packaging cell and target cell cultures, respectively, before, after, or at the same time that retrovirus is applied to the cultures according to the methods of the present invention (with the limitations as disclosed herein). It is further contemplated herein that, while a glucocorticoid receptor agonist should be added to packaging cells to achieve an increase in viral titer, a glucocortiocoid receptor antagonist may be added to packaging cell and/or to target cell cultures and cause an increase in retroviral infectivity, e.g., a glucocorticoid antagonist (mifepristone or other) may be added to packaging cells at the time of viral propagation, or to a conditioned medium after its collection. A glucocorticoid agonist (dexamethasone or other) can be added after target cells are infected to increase protein expression levels of the delivered gene(s).

Thus, in some embodiments, either the packaging cells and the target cells, or both, may be cultured with one or more glucocorticoid receptor antagonists. Optionally, in some embodiments the packaging cells are cultured with a glucocorticoid receptor agonist prior to culture of the target cells with a glucocorticoid receptor antagonist, such that culture with glucocorticoid receptor agonist and glucocorticoid receptor antagonist occurs in a step-wise, or sequential fashion. However, it is understood that antagonist and agonist should not be cultured together to packaging cells.

Retrovirus may be applied to cultures of target cells according to conventional methods. These include, for example, the possibility that target cultures may be cultured with retrovirus-containing culture medium (“conditioned medium”) obtained from packaging cell cultures, or cultured with retrovirus that has been obtained from the conditioned medium, for example, by ultracentrifugation or column concentration or other conventional means of concentrating or harvesting the retrovirus particles for infection. It is also possible that the method of the present invention may be used with retrovirus that has been obtained from conditioned medium according to conventional methods and frozen for future application to target cell cultures.

Packaging cells suitable for use with particular retroviral vectors and delivery systems are familiar to one of skill in the art and include, for example, GP2-293, PT67, AmphoPack 293, EcoPack 293, 293RTV and other in addition to those described in the Examples provided hereinbelow. These and other packaging cell lines are available commercially, e.g., from e.g., Orbigen, Inc. (San Diego, Calif.).

As used herein, a “transgene” is understood to mean an exogenous gene introduced into the genome of another organism, e.g., by retroviral delivery.

Target cells, i.e., cells into which a nucleic acid can be delivered by a retrovirus, may encompass any desired, available cell type and may be of various origin, e.g., rat, mouse or human. For purposes of gene therapy, these cells may be autologous or non-autologous to the patient. Examples of these “target cells” include but are not limited to epithelial cells, including lung epithelial cells, endothelial cells, including vascular endothelial cells, keratinocytes, fibroblasts, muscle cells, including vascular smooth muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

Retroviral expression systems and reagents and materials for the propagation of retrovirus, including but not limited to cell culture reagents, growth medium, supplements, materials and culture conditions for the culture of various packaging cells and target cells for use with the methods of the present invention are familiar to one of skill in the art. All may be easily obtained from commercial suppliers. Similarly, various cell culture conditions, e.g., degree of cell confluence, cell density, incubation times, length of time for sufficient retroviral propagation, infection and integration, etc. best suited for carrying out the methods of the present invention are familiar to one of skill in the art, or may be easily ascertained by routine experimentation.

In addition to the separate culture of packaging cells and target cells, e.g., as described in the examples disclosed herein, retroviral propagation, infection and gene delivery may also be performed by co-cultivating packaging and target cells, e.g., by plating target cells on a lawn of packaging cells.

This present invention also covers the injection of packaging cells or concentrated virus into animals or humans in vivo followed by the administration of a glucocorticoid receptor antagonist (such as mifepristone or other) to improve target cell infection. This application also covers the injection of infected target cells into animals or humans in vivo with the subsequent administration of a glucocorticoid receptor agonist (dexamethasone or other) to increase the expression level of delivered genes.

“Conditioned medium” as used herein refers to culture medium harvested from cultures of packaging cells which comprise virus which has been propagated therein.

As used herein, “increasing target cell infectivity of retroviruses” and “increasing retroviral infectivity” have similar meanings, and refer to an increase in the ability of a retrovirus to infect a target cell.

“Time sufficient to achieve desired viral titer” as used herein is understood to refer to an amount of time necessary to propagate an amount of virus sufficient to result in a desired percentage of infected target cells. Such time and titers are known in the art, or may be determined by one of skill in the art according to conventional methods and without undue experimentation.

As used herein, a “desired viral titer” refers to a titer of virus sufficient to achieve a desired percentage of infected target cells. Desired titer and percentages may vary depending on the virus, cell types and/or nucleic acid to be delivered, and may be determined by one of skill in the art according to conventional methods and without undue experimentation.

As contemplated herein, retroviruses for use in the methods of the present invention include viruses of the family Retroviridae, particularly Gammaretroviruses, e.g., Murine leukemia virus, Murine Stem Cell Virus, Feline leukemia virus and others. Lentiviruses, e.g., Human, Simian and Feline immunodeficiency viruses may also prove suitable. In particular embodiments of the present invention the retrovirus is a Gammaretrovirus.

As familiar to one of skill in the art, the classification of retroviruses is arranged by computer analysis of genomes, based primarily on comparisons of the size and morphologic characteristics of the viral genome. All gamma-retroviruses have similarities in genome organization, but each possesses individual characteristics. For example, Moloney murine leukemia virus (MMLV) based vectors are produced by replacing the viral genes required for replication with the desired genes to be transferred and along with other vectors are currently used as gene transfer vehicles, J. Moloney, J. Natl. Cancer Inst. (U.S.) 24, 933 (1960). The MSCV (Murine Stem Cell Virus) is an important derivative of MMLV that has been optimized for introducing and expressing target genes into pluripotent cell lines, including murine and human hematopoietic embryonic stem cells, and embryonal carcinoma cells which are usually resistant to retroviral infection. The friend murine embryonic-stem cell virus (FMEV) which is a hybrid viral backbone [friend mink cell focus-forming (FMCF)/murine embryonic stem cell virus (MESV)] was also designed to optimize infection and protein expression in primary hematopoietic cells. These last two retroviral backbones can be used to infect the same types of cells as MMLV.

Corticosteroids are well known in the art and include mineralocorticoids and glucocorticoids. Glucocorticoids are anti-inflammatory agents commonly used to treat diseases associated with an overactive immune system, such as allergies, asthma, autoimmune diseases and sepsis. They also interfere with some of the abnormal mechanisms in cancer cells, so they are used in high doses to treat cancer.

Glucocorticoids cause their biological effects by binding to the glucocorticoid receptor. This type of receptor is activated by ligand binding. After a hormone binds to the corresponding receptor, the newly-formed receptor-ligand complex translocates itself into the cell nucleus, where it binds to glucocorticoid response elements in the promoter region of the target genes resulting in the regulation of gene expression.

As used herein, the term corticosteroids includes “synthetic corticosteroids” or “synthetic corticosteriod analogs” which includes compounds which mimic the activity of the natural hormones, e.g., cortisone and hydrocortisone. These include, for example, prednisone and prednisolone, dexamethasone and mifepristone, Org 34517 (Schering-Plough), fludrocortisone, betamethasone and trimacinolone, among others familiar to one of skill in the art.

Dexamethasone is a well known anti-inflammatory and immunosuppressant drug, with a potency of about 20-30 times that of the naturally occurring hormone hydrocortisone and 4-5 times that of prednisone. Dexamethasone is well known to one of skill in the art and is commercially available from a wide variety of chemical supply companies.

As the term is used herein, “dexamethasone” includes analogs and derivatives of this drug. These include, for example, cortisol, cortisone, prednisone, prednisolone and methylprednisolone.

According to the methods of the present invention, a glucocorticoid receptor agonist, e.g., dexamethasone or an analog thereof, is used in an amount sufficient to cause an increase in viral titer, and thus an increase in retroviral infectivity, greater than that obtained in cultures maintained in the absence of this agonist. Suitable amounts are easily discernable by one of skill in the art without undue experimentation, and generally range, for example, from about 0.010 to about 1000 nanomoles.

Mifepristone (also known as RU486, or RU38.486, or 17-beta-hydroxy-1′-beta-(4-dimethylaminophenyl)-17-alpha-(1-propynyl)-estra-4,9-dien-3-one), or 11-beta-(4-dimethylaminophenyl)-17-beta-hydroxy-17-alpha-(1-propynyl)-estra-4,9-dien-3-one) is a synthetic corticosteroid which can bind to the glucocorticoid receptor, typically with high affinity, and thus inhibit the biological effects initiated/mediated by the binding of any cortisol or cortisol analogue to the glucocorticoid receptor. See, e.g., U.S. Pat. No. 7,402,578.

As the term is used herein, “mifepristone” includes analogs and derivatives of this drug. These include, for example, Org 34517. See also, e.g., Bachmann C G et al., Neuropsychopharmacology 2003 June; 28(6):1056-67.

According to the methods of the present invention, a glucocorticoid receptor antagonist, e.g., mifepristone or analog thereof, is used in an amount sufficient to cause an increase in target cell infectivity (or sensitivity to infection), and thus an increase in retroviral infectivity, greater than that obtained in cultures maintained in the absence of this antagonist. Suitable amounts are easily discernable by one of skill in the art without undue experimentation, and generally range, for example, from about 2 nanomoles to about 20 micromoles.

As contemplated herein, the methods of the present invention may be used to enhance gene delivery (e.g., administration of a nucleic acid to a subject for integration into the genome of the subject) and thus improve currently available methods employed in gene therapy. The technique should provide for the stable transfer of the nucleic acid to the cell, so that the nucleic acid is expressible by the cell and preferably heritable and expressible by its cell progeny.

Briefly, delivery of a nucleic acid into a patient by gene therapy may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy, and are familiar to one of skill in the art.

Typically, as contemplated herein, when directly administered in vivo, the nucleic acid is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral vector (see, e.g., U.S. Pat. No. 4,980,286 and others mentioned infra).

In some cases, the transfer of nucleic acid to a cell by use of a retroviral vector includes the transfer of a selectable marker to the cells. The cells are then placed under selection to isolate those cells that have taken up and are expressing the transferred gene. Those cells are then delivered to a patient. Typically, the nucleic acid is introduced into a cell prior to administration in vivo of the resulting recombinant cell. Suitable markers include, e.g., fluorescent markers, e.g., GFP, YFP DsRed, mCherry or enzymatic markers, e.g., β-Galactosidase, alkaline phosphatase, horseradish peroxidase, or any other typically used markers for gene delivery widely described in the literature and familiar to one of skill in the art.

As discussed above, target cells into which a nucleic acid can be delivered by a retrovirus for gene therapy encompass any desired, available cell type and will depend on the particular medical condition to be ameliorated. The cells may be autologous or non-autologous to the patient.

The resulting recombinant target cells can be delivered to a patient by various methods known in the art. For example, recombinant epithelial cells may be subcutaneously injected into a patient, or applied as a skin graft onto the patient, while recombinant blood cells (e.g., hematopoietic stem or progenitor cells) may be administered intravenously. The amount of cells and types of recombinant cells that may be treated according to the methods of the present invention envisioned for use depends on the desired effect, patient state, etc., and can be determined by one skilled in the art.

The present invention also includes kits which comprise components for carrying out the methods of the present invention. Such retroviral transfection kits may be specifically designed not only to increase viral production by packaging cell lines, but to improve target cell infectivity, which is a goal of many investigators using retroviral vectors. As contemplated herein, these kits may comprise one or more corticosteroids, e.g., a first and second corticosteroid, wherein the first corticosteroid is a glucocorticoid receptor agonist, and wherein the second corticosteroid is a glucocorticoid receptor antagonist. The kits may further comprise one or more additional retroviral infectivity enhancers and transfection reagents. In another particular embodiment the kit comprises mifepristone, or analogs or derivatives thereof, in amounts sufficient to increase retroviral target cell sensitivity to retroviral infection. In another particular embodiment, the kit comprises dexamethasone and mifepristone, or analogs or derivatives thereof, in amounts sufficient to increase retroviral titer, or target cell sensitivity to retroviral infection, respectively, and which when applied according to the methods of the present invention can increase target cell infectivity of retroviruses.

All publications cited herein are indicative of the level of skill of those skilled in the art to which this invention pertains. All these publications are herein incorporated by reference in their entirety to the same extent as if each individual publication were specifically and individually indicated to be incorporated by reference.

### EXAMPLES

The data and figures provided below as Examples 1-4 were previously disclosed in Solodushko V, Bitko V, Fouty B. Dexamethasone and mifepristone increase retroviral infectivity through different mechanisms. Am J Physiol Lung Cell Mol Physiol 2009; 297: L538-545, the contents of which are incorporated by reference herein in its entirety. Materials and methods for Examples 1-4 are provided below:

Materials: Dexamethasone (D4902), Mifepristone (M8046), hexadimethrine bromide (H9268), and purified monoclonal mouse anti-β-actin antibody (AC-15, horseradish peroxidase (HRP) conjugated) were all purchased from Sigma (St. Louis, Mo.). Mouse monoclonal anti-Green Fluorescent Protein (GFP) antibody (B-2, sc-9996, HRP conjugated) was purchased from Santa Cruz Biotechnology (Santa Cruz, Calif.). Dulbecco's Modified Eagle Medium (DMEM), trypsin-ethylenediaminetetraacetic acid (EDTA) and L-glutamine were all purchased from Gibco (Grand Island, N.Y.). Fetal bovine serum (FBS) was purchased from Atlanta Biologicals (Lawrenceville, Ga.). HyBond-P membrane was purchased from Amersham (Buckinghamshire, England). SuperSignal West Dura was purchased from Pierce (Rockford, Ill.). Charcoal stripped FBS (GTX73240) was purchased from GeneTex. FuGENE 6 Transfection Reagent (11 814 443 001) was purchased from Roche Diagnostics (Indianapolis, Ind.).

Cell cultures: Phoenix (ecotropic) packaging cells were purchased from Orbigen, Inc. (San Diego, Calif.). Rat pulmonary microvascular endothelial cells (PMVEC) were obtained from our cell culture core at the University of South Alabama (King J et al. Structural and functional characteristics of lung macro and microvascular endothelial phenotypes. Microvascular Res. 2004, March, 67(2). 139-151). Phoenix and PMVEC were cultured in DMEM, 10% FBS, 2 mmol L-Glutamine and used for experiments at passages 4-9. For some experiments charcoal stripped FBS was used in place of normal FBS. All cells were grown in humidified incubators at 37° C. in 5% CO₂.

Vectors and delivery systems: pBMN-GFP, a retroviral vector that expresses GFP driven by the MMLV's promoter in Phoenix and other cells was purchased from Orbigen, Inc. (San Diego, Calif.). Packaging cells were transfected with pBMN-GFP using FuGENE 6 reagent following manufacture instructions. Stably transfected Phoenix cells were selected by GFP expression using flow cytometry and then used for experiments. For virus propagation, packaging cells were cultured in growth medium for at least 12 hours. Virus was collected with cultured medium, filtered, supplemented with hexadimethrine bromide (polybrene) to a final concentration of 4 μg/ml and applied directly to target cells. The level of viral RNA in collected medium was determined by quantitative PCR using iScript One-Step RT-PCR kit (with SYBR Green, Bio-Rad, 170-8893) using primers for the GFP region of the viral RNA following manufacture instructions.

Western blotting: Cells were harvested, lysed, resolved in SDS-PAGE, and probed with HRP conjugated antibodies overnight. Positive antibody reactions were visualized using SuperSignal chemiluminescence detection system (Pierce) according to the manufacturer's instructions.

Flow cytometry analysis: Cells were harvested by 0.05% trypsin/0.53 mM EDTA digestion, washed, re-suspended in cultured medium and analyzed directly by FACScan in the University of South Alabama Flow Cytometry Core.

Statistical analysis: Data are expressed as means±SE. Cell growth, infectivity and changes in fluorescent proteins expression were compared using ANOVA combined with Fisher post hoc analysis, with a P value<0.05 considered significant.

### Example 1

### Dexamethasone Enhances Expression of Viral Proteins and Increases Retroviral Titer

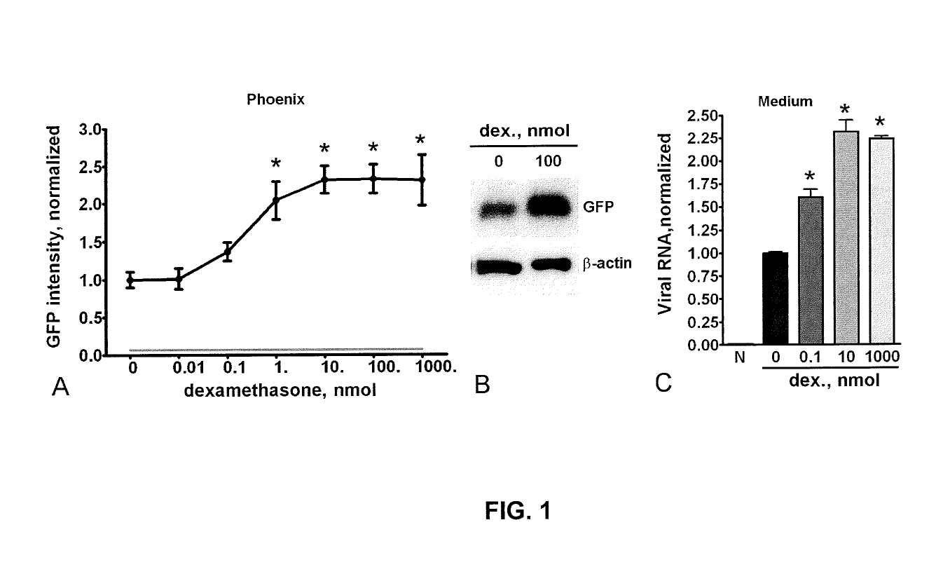

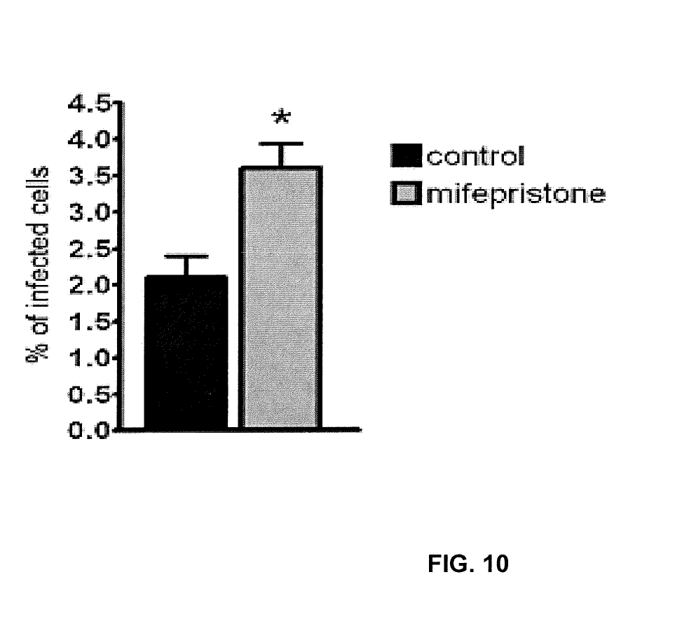

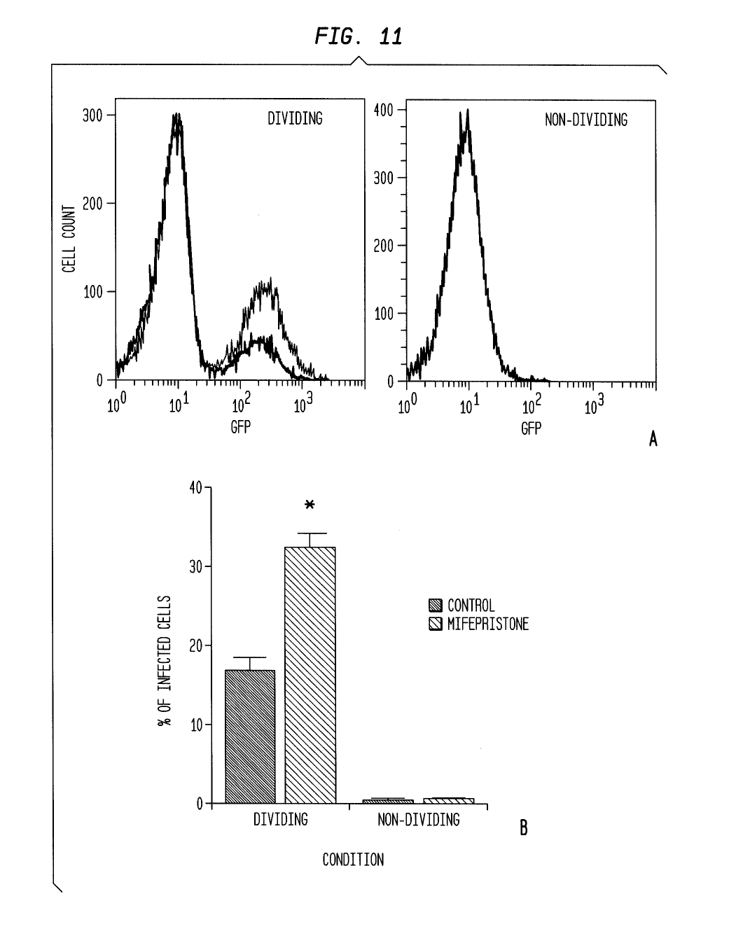

We first tested the ability of dexamethasone to increase retroviral protein production in Phoenix cells with integrated pro-viruses using GFP as a reporter. We cultured cells for 36 hrs in DMEM supplemented with 10% FBS and varying concentrations of dexamethasone. We then analyzed the Phoenix cells for GFP expression by flow cytometry. As shown in FIG. 1A, dexamethasone at concentrations of 10 nmol and higher, more than doubled GFP levels in these cells. We confirmed this by Western blot analysis of cell lysates obtained from vehicle and dexamethasone-treated cells (FIG. 1B). To determine if this increase in GFP expression correlated with an increase in viral titer in the supernatant, we performed quantitative RT-PCR on the conditioned medium using primers for the GFP region of the viral RNA. Viral titer increased in a dose-dependent manner reaching a plateau at 10 nmol of dexamethasone (FIG. 10) suggesting that at this concentration, the pro-viral LTR (long terminal repeat) promoter is maximally activated by dexamethasone.

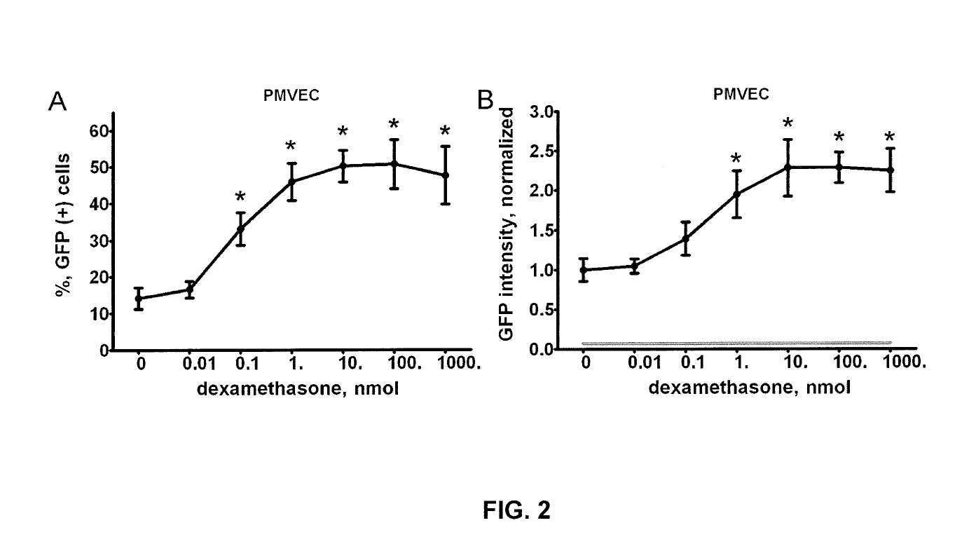

While this result demonstrated that dexamethasone could stimulate the retroviral LTR promoter of the pro-virus, the true measure of retroviral assembly and infectivity is the ability to infect other (target) cells. Therefore to test whether dexamethasone increased viral production (and infectivity), ecotropic Phoenix cells were grown with increasing concentrations of dexamethasone for 36 hours to about 50% confluence. Conditioned medium was then collected and directly applied to rat pulmonary microvascular endothelial cells (PMVEC). After 12 hours the conditioned medium was replaced with fresh medium with the same concentration of dexamethasone for an additional 72 hours. Cells were then collected and analyzed for GFP expression using flow cytometry. The percentage of GFP-positive (i.e. infected) PMVEC increased as the amount of dexamethasone in the conditioned medium increased. This occurred in a dose-dependent fashion and reached a plateau at 10 nmol where 45% of rat PMVEC were infected (FIG. 2A).

To assess whether dexamethasone had the same stimulatory effect on the LTR promoter of the pro-virus integrated into the target cells that it did on packaging cells (FIG. 1A), the expression level of GFP in infected cells was also measured. FIG. 2B demonstrates that dexamethasone stimulated the LTR promoter of the provirus integrated into the target cells in the same dose-dependent fashion demonstrated in the packaging cells. Dexamethasone had no effect on cell growth over 7 days in either retrovirus producing or target cells (data not shown).

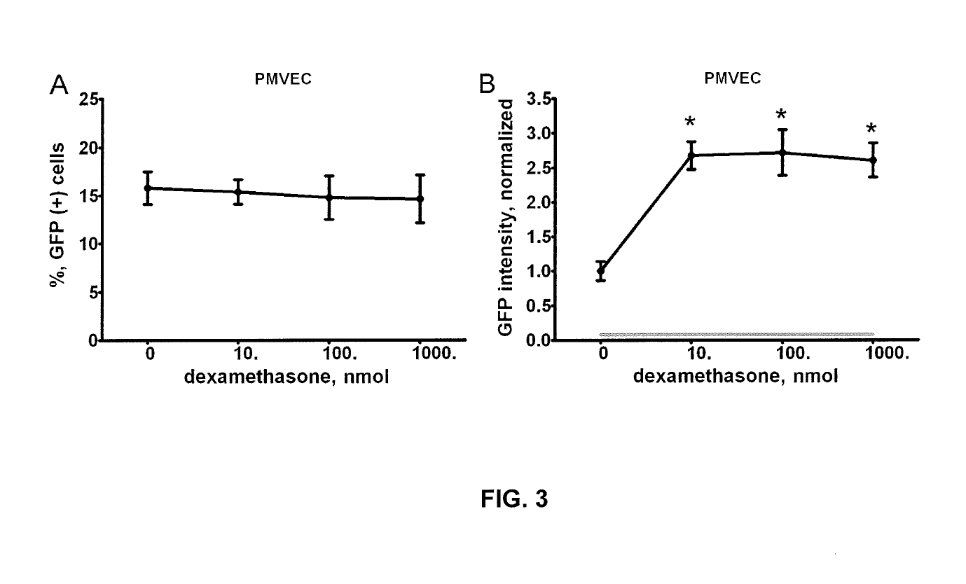

To determine whether dexamethasone itself increased retroviral activity by facilitating viral infection, we took conditioned medium from Phoenix cells grown without dexamethasone and then added increasing concentrations of dexamethasone. We applied the retroviral-conditioned medium to rat PMVEC for 12 hours. The viral supernatants were then replaced with fresh culture medium with the same dexamethasone concentrations for the next 72 hours. Cells were then analyzed by flow cytometry to determine the percent of GFP-positive cells. Supplementation of conditioned medium with dexamethasone (in the absence of Phoenix cells) did not increase the percent of infected target cells (FIG. 3A). Consistent with our previous data, however, dexamethasone did increase the intensity of GFP expression in those cells that were infected (FIG. 3B). This indicates that the addition of dexamethasone at the time of virus propagation, but not after medium collection, increased viral activity. The addition of dexamethasone to collected medium did not alter infection efficiency, but it did boost expression of viral proteins in those target cells that were infected.

### Example 2

### Reducing Steroid Hormones in FBS Decreases Activation of LTR Promoter and Reduces Viral Titer

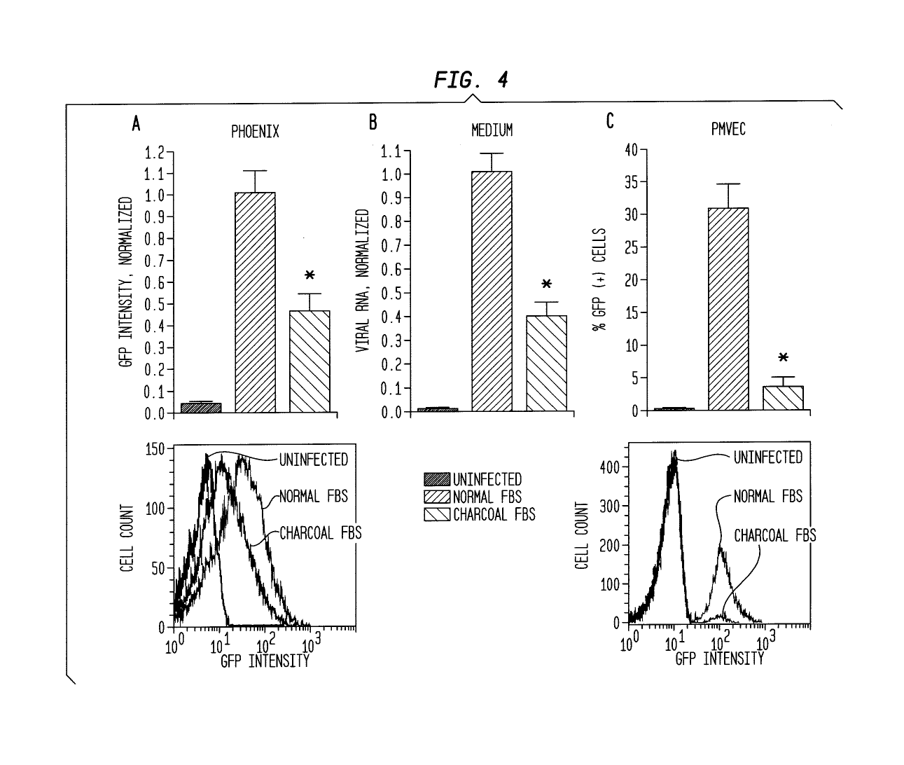

Since steroid hormones present in serum may activate the LTR promoter at baseline (i.e. before the addition of dexamethasone), we examined whether reducing these steroids in serum would decrease virus propagation and infectivity. We grew Phoenix cells with an integrated pro-virus expressing GFP to maximum confluence in DMEM supplemented with 10% charcoal-stripped FBS (which has a reduced steroid hormone content) (8) for 72 hours. GFP expression in these cells was less than half that of cells exposed to regular FBS (FIG. 4A). Using primers for the GFP region of the viral RNA we performed quantitative RT-PCR of the conditioned medium and found a decrease in retroviral RNA levels in collected medium obtained from packaging cells grown in charcoal-stripped FBS. The viral titers correlated directly with the GFP levels in Phoenix cells (FIG. 4B). The conditioned medium collected from packaging cells grown in charcoal-stripped FBS demonstrated about a seven-fold decrease in ability to infect target cells compared to medium collected from packaging cells grown in normal serum (FIG. 4C). Charcoal-stripped FBS had no adverse effect on the growth or viability of the packaging cells over 10 days nor was it detrimental to the target cells during a 12-hour incubation (data not shown).

### Example 3

### The Glucocorticoid Receptor Antagonist, Mifepristone, Increases Target Cell Infectivity Independent of Viral Titer

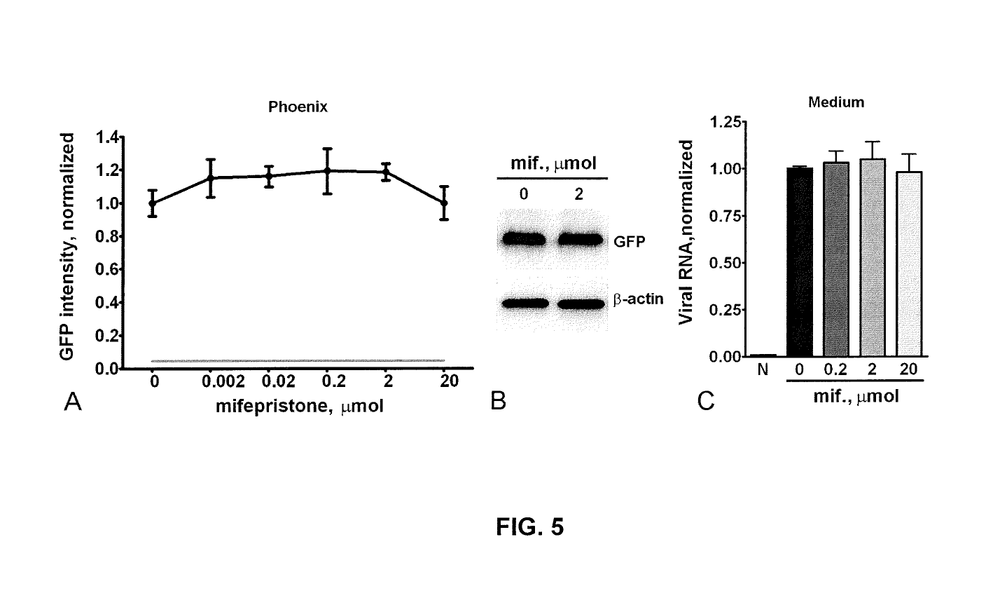

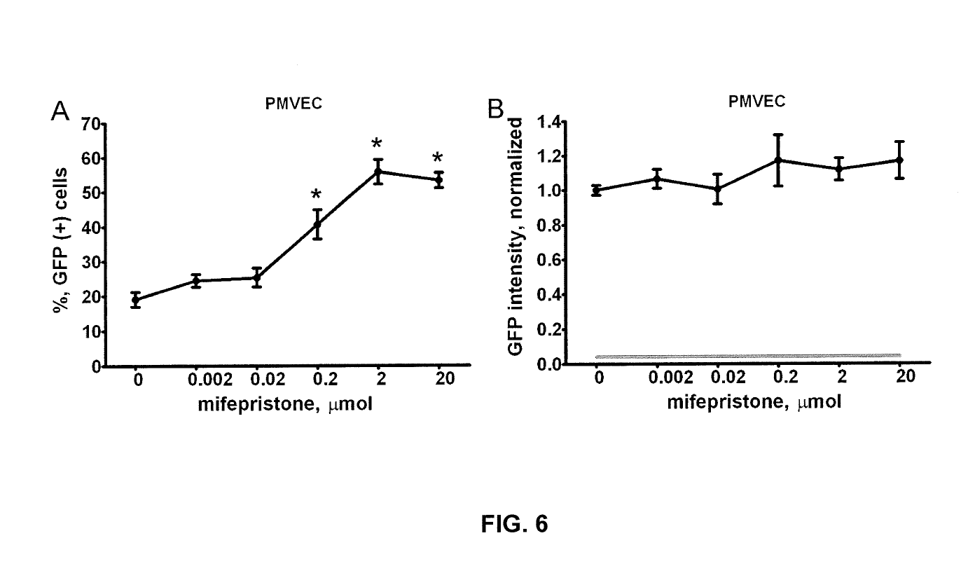

The retroviral LTR promoter is known to have hormone response elements (5, 12) and it appeared that this promoter was stimulated not only by dexamethasone, but by steroid hormones within FBS that can be removed by charcoal (FIG. 4). Mifepristone (RU 486) is a glucocorticoid (and progesterone) receptor antagonist that can act as an abortifacient (4). We tested the effect of varying concentrations of mifepristone on retroviral production in Phoenix cells. We grew cells to 50% confluence (as described above) in the presence of increasing concentrations of mifepristone (0-20 μmol), but without dexamethasone. Conditioned medium was then collected and applied directly to PMVEC. After 12 hours the conditioned medium was replaced with fresh medium with the same concentration of mifepristone for an additional 72 hours. Cells were then collected and analyzed for GFP expression. In contrast to the effects seen with dexamethasone, mifepristone had no effect on the GFP level in the packaging cells (FIG. 5A) suggesting that it did not affect (either positively or negatively) the LTR promoter activity of the integrated pro-virus under these conditions. Western blot analysis of the cell lysates obtained from vehicle- and mifepristone-treated cells confirmed this observation (FIG. 5B). To determine whether mifepristone had increased viral titer without directly stimulating the LTR promoter of the integrated pro-virus, the viral level in the collected medium was analyzed using primers for the GFP region of the viral RNA. Mifepristone had no effect on viral levels in the medium (FIG. 5C).

To determine whether mifepristone could change viral infectivity, the conditioned medium from these Phoenix cells was added to rat PMVEC for 12 hours and the percentage of GFP-positive cells determined 72 hours later. Surprisingly, the presence of mifepristone in the medium increased the susceptibility of target cells to viral infection even though it had no effect on viral titers (FIG. 6A). Mifepristone at 2 μmol and higher concentration more than doubled the percentage of infected cells. Similar to its effect on the packaging cells, mifepristone did not alter GFP expression in infected target cells indicating that unlike dexamethasone, it did not stimulate the pro-viral LTR (FIG. 6B).

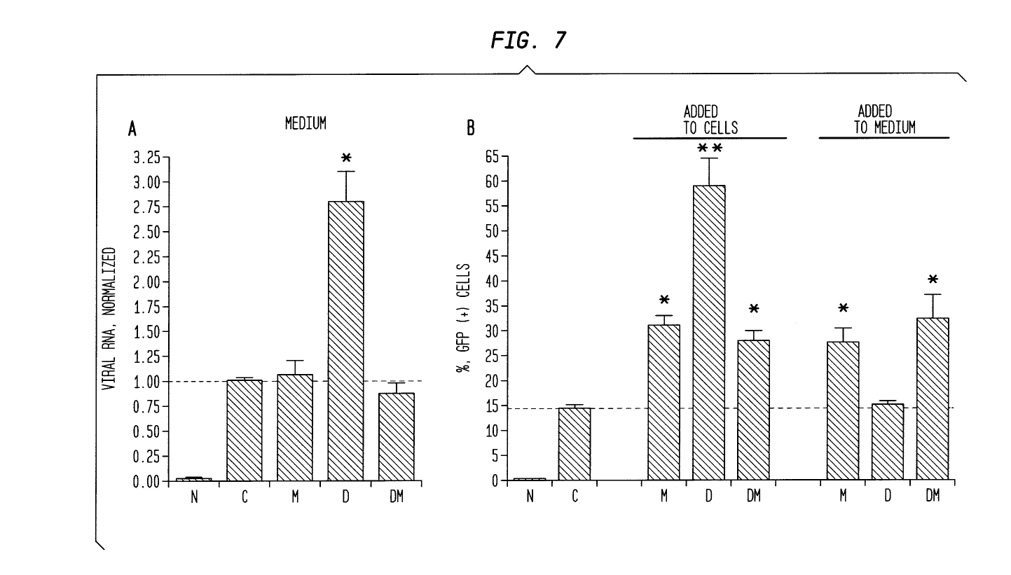

We next tested the effect of adding both dexamethasone and mifepristone to the packaging cell line at the time of viral propagation. Consistent with our previous results, dexamethasone elevated viral RNA level in the medium from Phoenix cells, whereas mifepristone did not (FIG. 7A). When both dexamethasone and mifepristone were added to the packaging cells at the same time, however, mifepristone completely inhibited the enhancing effect of dexamethasone on virus production (FIG. 7A). This result is likely explained by mifepristone's ability to compete with dexamethasone for binding sites on the glucocorticoid receptor leading to decreased activation of the LTR promoter (5, 23). FIG. 7B demonstrates that mifepristone increased target cell infectivity to a similar degree whether it was added to the packaging cell line at the time of viral propagation or to the conditioned medium after its collection. In contrast, dexamethasone must be added to the packaging cell line to be effective. When both dexamethasone and mifepristone were added to the packaging cell line, the infectivity mirrors that seen with mifepristone alone.

To determine whether either mifepristone or dexamethasone were increasing GFP intensity and expression through direct effects on GFP stability or fluorescence intensity, we repeated these experiments using two other retroviruses, one in which GFP was replaced by DsRed and another in which it was replaced by mCherry. We found a similar effect on target cell infectivity and fluorescence intensity per cell indicating that non-specific effects of dexamethasone or mifepristone on reporter gene stability were not the explanation for our results (data not shown).

### Example 4

### Sequential Addition of Dexamethasone and Mifepristone Yields Highest Target Cell Infectivity Rate

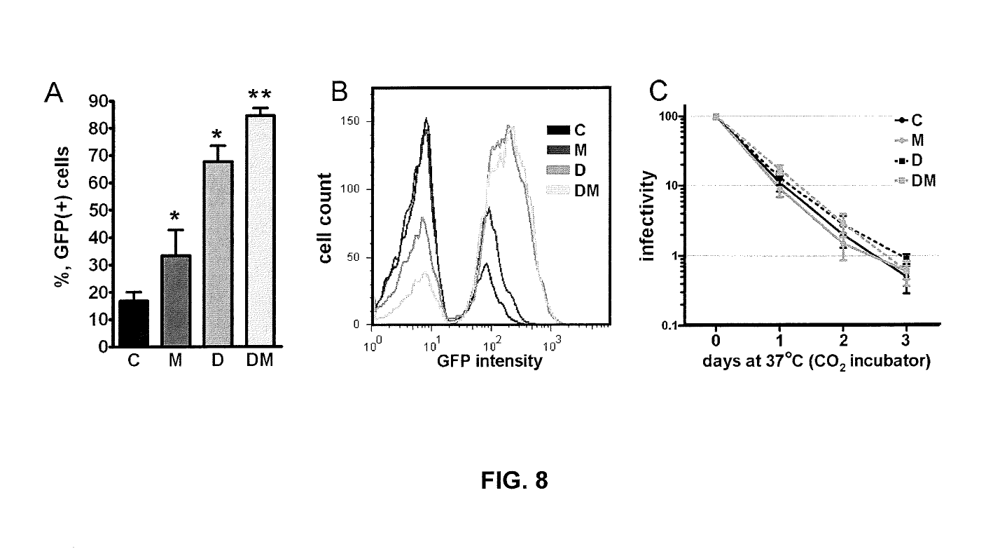

The above data suggested that dexamethasone increased viral titers when added to packaging cells while mifepristone increased the infectivity of target cells through another mechanism that was independent of viral titer. This suggested that the highest rate of infectivity should occur when dexamethasone was incubated with packaging cells without mifepristone, followed by the addition of mifepristone to the collected conditioned medium before incubation with target cells. To test this, ecotropic retroviruses expressing GFP were propagated in medium with or without 100 nmol dexamethasone. After collection, the conditioned medium was added to rat PMVEC with or without 2 μmol of mifepristone. After 12 hours of infection the medium was replaced with fresh medium and target cells were analyzed for GFP after 72 hours. FIG. 8A demonstrates the percent of infected cells under each of these different conditions. The stepwise addition of dexamethasone and mifepristone increased retroviral infectivity the greatest, from 16% in untreated cells to 85% in sequentially treated cells. FIG. 8B is a representation of our flow cytometry data that demonstrates that the addition of dexamethasone to the packaging cell line increased both the number of infected target cells (y-axis) and the intensity of GFP within those infected cells (x-axis) whereas the addition of mifepristone increased only the number of infected target cells.

To demonstrate that mifepristone and/or dexamethasone did not increase infectivity by prolonging viral particle survival, we tested the infectivity of conditioned medium with dexamethasone or mifepristone after several days of incubation in a CO₂ incubator at 37° C. FIG. 8C demonstrates that the number of positive (infected) target cells decreased by about 85-90% over each 24 hour time period compared to fresh conditioned medium. This decline was not attenuated by the addition of either dexamethasone or mifepristone.

Based on the above findings, we conclude that 1) dexamethasone can stimulate the LTR promoter of integrated pro-viruses in different cell types, 2) dexamethasone increases the viral titer of (Phoenix) packaging cell lines and this results in an increase in target cell infectivity, 3) mifepristone can increase target cell infectivity independent of retroviral titer, and 4) the sequential addition of dexamethasone and mifepristone is the most effective strategy to increase target cell infectivity in our system.

The family Retroviridae comprises a variety of enveloped RNA viruses, such as endogenous retroviruses, leukemia viruses, and HIV-1. Their replication requires reverse transcription of the viral RNA into linear double-stranded DNA and the subsequent integration of this DNA into the genome of the cell (provirus). To make new virus, the viral DNA genome must first be copied back to RNA using a host enzyme, RNA polymerase II. The LTR region is the control center for gene expression. All of the requisite signals for gene expression are found in the LTR. Retroviral LTRs contain elements that control the level and tissue-specificity of transcription and include a hormone response element responsible for stimulating retroviral transcription in the presence of specific steroid hormones. These hormones control many physiological processes by activating specific receptors that act as transcription factors.

In vivo many LTR promoters can be activated by glucocorticoids and progestins through the interaction of the hormone-receptor complex with hormone response elements. Glucocorticoids regulate transcription of provirus through the binding of a glucocorticoid receptor to the glucocorticoid response element that has been described for a number of retroviruses (3, 9, 10, 14, 17, 20, 24), including human immunodeficiency virus (HIV) (15, 18, 22, 25). Though the activation of the pro-viral promoter by dexamethasone or other steroids has been described (1, 6, 7, 11, 19), the demonstration that this leads to an increase in the formation of active viral particles and ultimately to an increased infectivity of target cells has not. Here we show that dexamethasone can not only regulate viral promoters, but also increase target cell infectivity by increasing viral titers in the medium of packaging cells.

Though the biological significance of steroid hormones in retroviral cycling is not clear, the advantage of stimulating retroviral production to obtain higher titer virions for gene delivery is clear. In most protocols for generating retrovirus, the only source of hormones in cell culture is serum. Our experiments using charcoal-stripped medium to remove these steroids, indicate how important they are for viral production. We demonstrate that the addition of dexamethasone at just 10 nmol concentration, however, can markedly increase the viral titer, without any evident adverse effects on cell proliferation or survival. Since the glucocorticoid receptor antagonist, mifepristone, partially blocks the stimulatory effect of dexamethasone on viral production in Phoenix cells, dexamethasone's effect likely requires binding to the glucocorticoid receptor.

MMLV is widely used as a DNA-delivery vehicle for gene therapy (2). It is a suitable model for studying retroviral function in response to dexamethasone and mifepristone. We were able to increase retroviral infection efficiency by first stimulating the viral promoter in Phoenix cells using dexamethasone and then by adding mifepristone to target cells at the time of virus infection. Each or both steps (using dexamethasone and/or mifepristone) may be included in a standard protocol for retroviral propagation and infection of target cells. Both steps are very easy to perform and did not cause any evident adverse effects on cell growth or survival in either the packaging or target cells. After the initial infection cycle, dexamethasone and mifepristone may be removed from the medium. Keeping dexamethasone in the medium after infection, however, did increase the expression level of target and reporter proteins. This may be especially useful if target protein(s) are unstable or difficult to transcribe and it may also facilitate cell analysis and sorting.

We used the pBMN-GFP retroviral system, with ecotrophic Phoenix packaging cells for viral propagation, and rat PMVEC as target cells. Since the actual infection efficiency may vary depending on cell type, we also performed infectivity studies on other host (target) cells including human glioblastoma cells (U87MG), rat pulmonary artery endothelial cells, rat pulmonary artery smooth muscle cells, and HeLa cells. We obtained qualitatively similar results. In addition, we demonstrated that dexamethasone and mifepristone had similar effects on ecotropic, amphotropic and VSV-G based pantropic systems. Because reporter protein levels can be determined by both protein translation and protein stability, we replaced GFP with DsRed or mCherry to show that the type of reporter gene did not alter our results.

We also demonstrate that mifepristone can enhance retroviral infectivity of target cells without increasing retroviral production in packaging cells. This unexpected effect of mifepristone can be used to improve target cell infectivity that is additive to increasing viral titer and can be used to increase the infectivity of already collected and stored (frozen) retroviral stocks. Based on these data, the mechanism through which this viral-independent effect occurs is not clear. Mifepristone may increase the activity of reverse transcriptase or it may facilitate transport of the newly transcribed viral DNA into the nucleus where it is integrated into the host genome.

This observation that mifepristone can increase the infectivity of target cells is not only important in improving target cell infectivity, but may have relevance to retroviral biology. Interest has been generated that mifepristone, acting as an antagonist of the glucocorticoid receptor, might block HIV replication within infected cells. A recent publication demonstrated that in a dose-dependent manner, mifepristone inhibited the viral protein R (Vpr)-mediated transactivation of HIV-1 LTR-driven gene expression in HeLa cells, raising hope that mifepristone might be a potential adjuvant therapy for HIV infection. (23) One publication published over a decade ago, however, demonstrated that mifepristone could stimulate HIV replication depending on cell type (26). Our results suggest that mifepristone might also increase the ability of retroviruses to infect target cells. Strategies using mifepristone to combat retroviral infections clinically, therefore, should be pursued cautiously.

Data from Examples 1-4 indicate that using dexamethasone and mifepristone in retroviral systems may significantly increase the infection efficiency of viral supernatants especially for situations in which baseline infectivity is low. While our results were performed in vitro, such a strategy may be useful to improve infection efficiency in vivo. In addition, the described effects of dexamethasone and mifepristone on retroviral promoter activity and infectivity, may be important for understanding the cellular mechanisms regulating retroviral behavior.

Taken together, the findings described above demonstrate that the synthetic anti-glucocorticoid and anti-progestin agent, mifepristone, increased gamma-retroviral infection efficiency in different target cells, independent of viral titer. As described in detail in the Examples 5-13 provided below, additional experiments were performed to examine the mechanism by which this may occur by studying the effect of mifepristone on different steps of viral infection (viral entry, viral survival, viral DNA synthesis and retrovirus integration into the host genome) in three distinct retroviral backbones using different virus recognition receptors. We also tested the potential role of glucocorticoid and progesterone receptors in mediating mifepristone's ability to increase gamma-retroviral infectivity. The data and figures provided below as Examples 5-13 were previously disclosed in V. Solodushko and B. Fouty, Mifepristone increases gamma-retroviral infection efficiency by enhancing the integration of virus into the genome of infected cells. Gene Therapy 2010, 1-9, (49) the contents of which are incorporated by reference herein in its entirety.

Materials and methods for Examples 5-13 are provided below:

Materials: Dexamethasone (D4902), Mifepristone (M8046), hexadimethrine bromide (H9268), propidium iodide (PI), RNase, and 5′-bromo-2′-deoxyuridine (BrdU) were all purchased from Sigma (St. Louis, Mo.). Dulbecco's Modified Eagle Medium (DMEM), trypsin-ethylenediaminetetraacetic acid (EDTA) and L-glutamine were all purchased from Gibco (Grand Island, N.Y.). Fetal bovine serum (FBS) was purchased from Atlanta Biologicals (Lawrenceville, Ga.). HyBond-P membrane was purchased from Amersham (Buckinghamshire, England). FuGENE 6 Transfection Reagent (11 814 443 001) was purchased from Roche Diagnostics (Indianapolis, Ind.). Org 34517 was kindly gifted by Schering-Plough. Progesterone receptor antagonist 11b-[4-(acetyl)phenyl)-4′,5′-dihydro-2′-ethyl-5′-methylenespiro[estra-4,9-dien-17b,4′-oxazole]-3-one (RTI # 7453-102) was synthesized by Dr. Chunyang Jin at RTI International following the previously published procedure (44).

Cell cultures: Phoenix Retroviral Expression System ecotropic and amphotropic packaging cells were purchased from Orbigen, Inc. (San Diego, Calif.). The Pantropic Retroviral Expression System (VSV-G based, with pantropic packaging cells) was purchased from Clontech (Palo Alto, Calif.). Rat pulmonary microvascular endothelial cells (PMVEC), were obtained from our cell culture core at the University of South Alabama (King J, et al. Structural and functional characteristics of lung macro and microvascular endothelial phenotypes. Microvascular Res. 2004, March, 67(2). 139-151). Human glioblastoma cells (U87MG), and human mammary carcinoma cells (T47D) were obtained from ATCC. All cells were cultured in DMEM, 10% FBS, 2 mmol L-Glutamine and used for experiments at passages 4-9. All cells were grown in humidified incubators at 37° C. in 5% CO₂. Cells were harvested by 0.05% trypsin/0.53 mM EDTA digestion, washed, re-suspended in cultured medium and analyzed directly by FACScan in the University of South Alabama Flow Cytometry Core. For cell cycle analysis cellular DNA was stained with 10 mmol/l of propridium iodide for 20 minutes as described elsewhere (48) and then analyzed by FACScan.

Vectors and delivery systems: pBMN-GFP (Orbigen, Inc., San Diego, Calif.), a retroviral vector that expresses GFP driven by the MMLV's promoter in Phoenix and other cells was used. In some experiments Murine Stem Cell Virus (MSCV) expressing mCherry and the hybrid retroviral vector Friend Murine Embryonic-stem cell Virus (FMEV) [for Friend Mink Cell Focus-forming (FMCF)/Murine Embryonic Stem cell Virus (MESV)](31) expressing DsRed2 were used in addition to MMLV. Two lentiviral vectors pLemiR (Open Biosystems, Huntsville, Ala.) and pLVX-DsRed-Monomer-C1 (Clontech Laboratories, Inc., Mountain View, Calif.) harboring turboRFP and DsRed as reporters were also used for comparison to gamma-retroviruses. Packaging cells were transfected with retroviral vectors using FuGENE 6 reagent following the manufacturer's instructions. For virus propagation, packaging cells were cultured in growth medium for 12-18 hours. Virus was collected with cultured medium, filtered, supplemented with hexadimethrine bromide (polybrene) to a final concentration of 4 μg/ml and applied directly to target cells.

Viral DNA measurement: The total DNA from infected cells was extracted by DNeasy Blood & tissue kit (Qiagen). The level of viral DNA in total DNA extract isolated from infected cells was determined by quantitative PCR using iScript One-Step RT-PCR kit (with SYBR Green, Bio-Rad, 170-8893) with primers for the GFP or the internal ribosome entry site regions of the viral DNA following the manufacturer's instructions. No reverse transcriptase was used in order to exclude contamination from a viral RNA signal. In integration studies, the total DNA isolated from infected cells was first resolved in a 0.8% agarose gel. 4 Kb DNA (the size of non-integrated viral DNA) and genomic DNA pools (>50 Kb) were excised. DNA from each fraction was extracted and analyzed for the presence of viral sequences by qPCR as previously described.

Statistical analysis: Data are expressed as means±SE. Changes in cell cycle profile, infectivity and changes in fluorescent protein expression were compared using ANOVA combined with Fisher post hoc analysis, with a P value<0.05 considered significant.

### Example 5

### Mifepristone Increases Infection Efficiency of Different Gamma-Retroviruses

The classification of retroviruses is arranged by computer analysis of genomes, based primarily on comparisons of the size and morphologic characteristics of the viral genome. All gamma-retroviruses have similarities in genome organization, but each possesses individual characteristics. Moloney murine leukemia virus (MMLV) based vectors are produced by replacing the viral genes required for replication with the desired genes to be transferred and along with other vectors are currently used as gene transfer vehicles (29). The MSCV (Murine Stem Cell Virus) is an important derivative of MMLV that has been optimized for introducing and expressing target genes into pluripotent cell lines, including murine and human hematopoietic embryonic stem cells, and embryonal carcinoma cells which are usually resistant to retroviral infection (30). The friend murine embryonic-stem cell virus (FMEV) which is a hybrid viral backbone [friend mink cell focus-forming (FMCF)/murine embryonic stem cell virus (MESV)] was also designed to optimize infection and protein expression in primary hematopoietic cells (31). These last two retroviral backbones can be used to infect the same types of cells as MMLV.

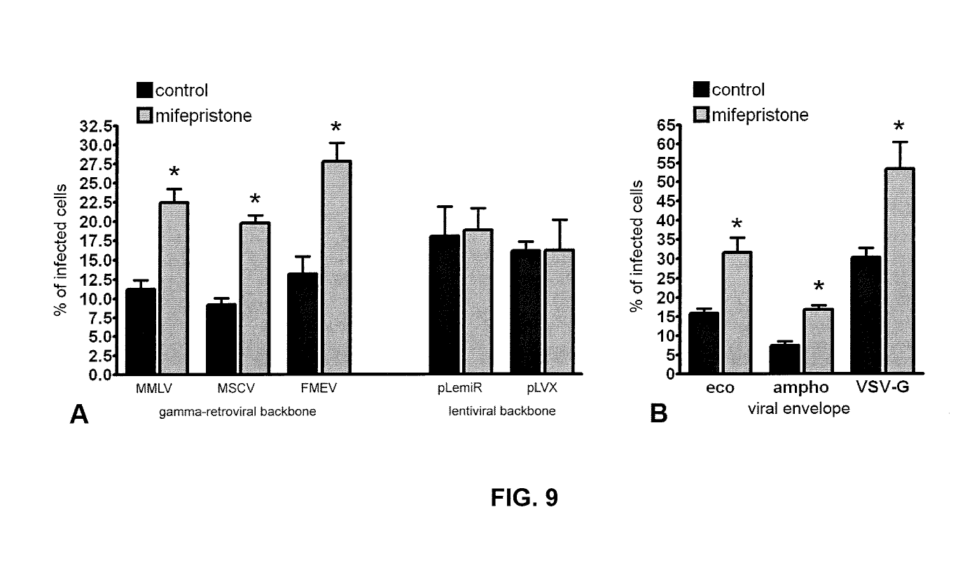

We investigated whether mifepristone could also increase the target cell infectivity of these popular gamma-retroviral systems. Gamma-retroviral backbones containing genes encoding fluorescent proteins as reporters were propagated in Phoenix ecotropic packaging cells. Viral-conditioned supernatant was collected and applied to target cells (rat pulmonary microvascular endothelial cells, PMVEC). FIG. 9A shows that mifepristone at 1 μmol/L concentration almost doubled the percentage of infected cells regardless of the viral backbone. This suggests that the ability of mifepristone to increase infectivity of gamma-retroviruses is not restricted to MMLV retroviral vectors, but also occurs in other popular gamma-retroviral gene delivery vectors. In contrast, mifepristone did not increase the target cell infectivity of lentiviral vectors (packed into VSV-G based viral particles).

### Example 6

### Mifepristone Increases Target Cell Infection Efficiency Independent of Viral or Target Cell Recognition Receptors

A viral receptor is conventionally defined as a component(s) on the cell surface to which a virus specifically binds and which may result in virus entry. Virus entry occurs following fusion between the retroviral envelope and the cellular plasma membrane (32). To determine if the type of viral receptor was critical to mifepristone's ability to increase target cell infectivity, we infected cells with MMLV packaged into viral particles with different envelope proteins—ecotropic, amphotropic, and VSV-G based pantropic. Ecotropic envelope proteins recognize the ecotropic receptor (mCAT1) (33) and can efficiently infect a broad range of mouse and rat cells. Amphotropic viral envelope proteins recognize the amphotropic receptor (Ram-1) and infect a broad range of mammalian cells (34). Viral entry with VSV-G pantropic envelope glycoproteins from the vesicular stomatitis virus does not require a cell surface receptor, but mediates viral entry through lipid binding and plasma membrane fusion (35).

As shown in FIG. 9B, the infection efficiency of rat PMVEC varied with the different viral particles, with VSV-G envelope glycoprotein demonstrating the greatest baseline infectivity while amphotropic viral particles demonstrated the least. While the baseline infectivity of ecotropic, amphotropic, and VSV-G based viruses was different (amphotropic receptors are less abundant in rodent cells compared to ecotropic ones, while VSV-G base viruses do not use any protein receptors for cell entry), mifepristone doubled the percentage of infected cells in each case. These results suggest that the ability of mifepristone to increase target cell infectivity is not dependent on the type of envelope protein expressed by the viral particles.

### Example 7

### Adding Mifepristone after Exposure to Retrovirus Still Increases Viral Infection Efficiency

Target cell entry is the first step of viral infection. This requires contact between the viral envelope and the cell membrane. Receptors on the viral envelope interact with complementary receptors on the cell membrane and this attachment causes the two membranes to remain in mutual proximity. To enter the cell through the phospholipid bilayer membrane, viral receptors attach to the receptors on the surface of the cell and either puncture the cell membrane or allow fusion of the viral envelope with the host cell. The virus's envelope then releases its contents into the cell (36).

To determine whether mifepristone increased retroviral infection efficiency by facilitating viral entry into target cells, PMVEC were infected for one hour in the absence of mifepristone. Cells were then washed (to remove the virus) and incubated in fresh medium for an additional hour. This allowed sufficient time for the virus to enter the cell, but not sufficient time to complete its infection cycle. Mifepristone (at 1 μmol/L concentration) was then added to the medium. Seventy-two hours later, target cells were trypsinized and analyzed for GFP expression by fluorescence activated cell sorting (FACS). FIG. 10 demonstrates that adding mifepristone after the initial infection had occurred (i.e. after the virus had been removed from the medium following its one hour incubation) also increased viral infection efficiency approximately two-fold (from 2.1 to 3.7%). The lower infection rate seen in FIG. 11 compared with FIG. 9 is due to the shorter retroviral incubation time (one versus 18 hours). This result suggests that the increased infection efficiency seen with mifepristone is not due to its effect on viral entry into target cells.

### Example 8

### Mifepristone Did not Promote Retroviral Infection in Non-Dividing Cells

Gamma-retroviruses and many other RNA viruses can only infect dividing cells because they must access the host DNA to replicate. The interior of the nucleus is separated from the cytoplasm by a double-layer membrane called the nuclear envelope. Embedded in the nuclear envelope are nuclear pores, which act like selective filters that allow diffusion of ions and molecules, but nothing larger (37). Gamma-retroviruses, therefore, cannot enter the nuclear compartment of non-dividing cells and integrate into the host genome. As cells undergo replication and division, the nuclear envelope becomes more permeable which allows greater cytoplasmic-nuclear trafficking and allows viruses to enter the nucleus. To determine whether mifepristone's ability to increase retroviral infection efficiency was due to its ability to infect non-dividing cells, we compared the infection efficiency in dividing (growing) and non-dividing (confluent) PMVEC with or without mifepristone. We grew cells to either 100% confluency (to induce cell arrest) or 15% confluency and then infected them with MMLV (at the same multiple of infectivity) in the presence of either 1 μmol/L of mifepristone or vehicle (EthOH). Seventy-two hours later, target cells were trypsinized and analyzed for GFP by FACS as previously described. FIG. 11 demonstrates that the retrovirus was only able to infect dividing cells and the presence of mifepristone had no effect on this.

### Example 9

### Mifepristone Does Not Prolong Survival of Non-Integrated Gamma-Retroviral Intermediates in Infected Cells

Since transduction of target cells by gamma-retroviruses is dependent on cell replication, it is important to know how long a virus that has successfully entered the target cell retains its ability to integrate. Some authors have demonstrated that if cells have not divided within 6 hours of infection, gene transfer does not occur (38). This suggests that retroviruses do not remain viable within the cell cytoplasm for a prolonged period of time. Other studies have shown that growth-arrested cells infected with retroviruses can undergo infection later; however, suggesting that some stable viral intermediates can persist in growth-arrested cells. This intermediate may be viral RNA, viral DNA (after reverse transcriptase) or a mixture of partially reverse-transcribed forms. The intracellular half-life of MMLV has been determined to be in the range of 5.5 to 7.5 hours (38, 39).

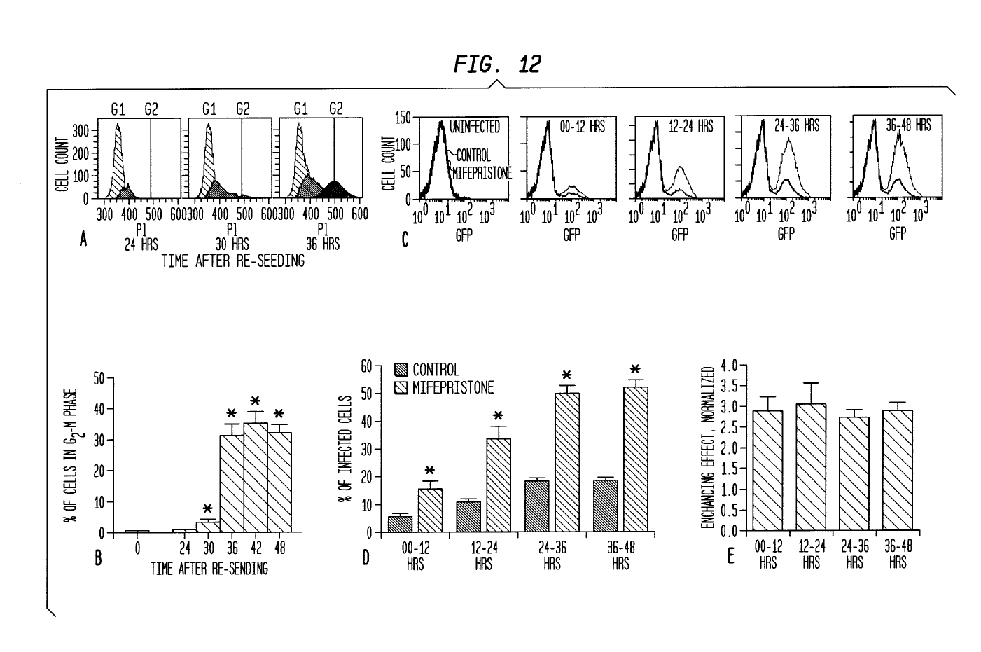

A potential mechanism by which mifepristone could increase the number of infected cells would be to prolong survival of the virus in infected but temporarily arrested cells. To evaluate this hypothesis we tested the ability of mifepristone to increase retroviral infectivity of growth-arrested cultured rat PMVEC as they re-entered the cell cycle over 48 hours. Rat PMVEC stop proliferating once they attain confluence and therefore are resistant to retroviral infection. We have previously demonstrated that once these growth-arrested confluent cells are re-seeded at lower confluency, they start to enter the S-phase approximately 24 hours and G2-M phase about 30 hours later. FIGS. 12A and 12B show the percentage of PMVEC entering G2-M phase at various time points after their reseeding from a confluent monolayer. Within this first 30 hours after reseeding, cells are not dividing and therefore while they may be able to take up virus, they are unable to complete an infection cycle by integrating it into the host genome.

PMVEC that were growth-arrested and then re-seeded at lower confluence as described above, were infected at various time points after reseeding in the presence of 1 μmol/L mifepristone or vehicle. Cells were incubated with virus for only 12 hours, but at different time points after re-seeding. Seventy-two hours later, the cells were analyzed for GFP using FACS.

FIG. 12D shows that cells infected immediately after re-seeding (i.e. 0 to 12 hours) demonstrated less infection efficiency compared to cells infected later (24 to 36 and 36 to 48 hours). Since arrested cells can take up the virus, but are unable to integrate it into the host genome, the decreased infection efficiency in cells infected immediately after re-seeding was likely due to the fact that by the time these cells began to divide (approximately 30 hours), most of the virus that had been taken up by the cells was already degraded. By the time the early-infected cells begin to divide (at about 30 hours), the virus has been degraded and is no longer capable of integration. As expected, mifepristone increased the infectivity rate at all time points (about 2.9 fold), but did not demonstrate an enhanced effect at the earlier time points (FIG. 12E). This suggests that mifepristone did not prolong the survival of non-integrated viral intermediates in these infected but temporarily non-dividing target cells. Had mifepristone prolonged viral survival, the earlier infected cells should have demonstrated a greater infectivity rate when compared to those cells infected later. FIG. 12E demonstrates that the increase in infectivity rate following incubation with mifepristone was similar at all time points, however. This correlates with our previously published results (28) demonstrating that mifepristone does not prolong viral viability in cell culture. FIG. 12C shows representative flow cytometry data of retroviral infectivity for all experimental conditions.

### Example 10

### Mifepristone Does Not Enhance Viral DNA Synthesis in Target Cells

Since mifepristone did not affect viral entry or survival in target cells but did increase the number of stably infected cells, we examined whether mifepristone stimulated other post-infection events in target cells including viral DNA synthesis (reverse transcriptase) or integration into the host genome. Reverse transcription—the transcribing of genetic information from RNA to DNA—is a hallmark of the retroviral replication cycle. The enzyme reverse transcriptase catalyzes this process and plays a critical role in viral cycling (40). To determine if viral DNA synthesis was stimulated by mifepristone, we performed quantitative PCR (qPCR) on total DNA isolated from target cells at various time points after infection (FIG. 13A). To better synchronize infection events, we exposed target cells to MMLV for only 1 hr in the presence of mifepristone or vehicle. After that, virus was removed from the medium. The viral DNA content in infected cells was measured by qPCR using primers to the GFP region of viral DNA. Mifepristone or vehicle was present in the medium from the beginning of infection until analysis (up to 7 days). Viral DNA content peaked six hours after infection and then began to decrease. There was no difference in viral DNA levels between mifepristone- and vehicle-treated cells in the first 6 hours suggesting that mifepristone did not affect viral DNA synthesis catalyzed by reverse transcriptase. Twenty four hours after infection the viral DNA content was decreased in all cells likely due to a combination of viral degradation and target cell proliferation resulting in the dilution of non-integrated viral DNA. The content of viral DNA in mifepristone-treated target cells was higher than that in vehicle-treated cells at 24 hours, a difference that persisted throughout the seven days of the experiment. This two-fold increase in viral DNA level observed in mifepristone-treated target cells at 3 days post infection closely correlated with the two-fold increase in the number of infected cells shown in FIG. 9 and to our earlier published results. Since the viral DNA content during log phase replication (0 to 6 hours) was not affected by mifepristone, it is unlikely that mifepristone increased target cell infectivity by stimulating viral reverse transcription.

### Example 11

### Mifepristone Enhances Viral Integration into Host DNA

While newly synthesized viral DNA can persist either as linear forms, one LTR circles, or two LTR circles for a period of time, ultimately this viral DNA must be either integrated (to complete an infection cycle) or degraded. If it is integrated into the host genome, viral DNA should be detectable within the genomic DNA immediately after incorporation. To examine whether mifepristone increased the amount of viral DNA integrated into the host genome, we infected cells with MMLV for 1 hour in the presence or absence of mifepristone, isolated total DNA from target cells at the time points described above and resolved the DNA on an agarose gel. 4 Kb DNA fragments (the size of non-integrated viral DNA) and genomic DNA (>50 Kb) were cut out and the DNA from each fraction was extracted and analyzed for the presence of viral sequences by qPCR. The level of free viral DNA (i.e. the viral 4 Kb DNA fragment) was markedly increased 6 hours after infection in both vehicle and mifepristone-treated cells and then declined towards baseline by 3 days (FIG. 13B). At no time did the mifepristone-treated cells demonstrate greater free viral DNA levels than vehicle-treated cells. This is consistent with our previous observation that mifepristone did not increase viral DNA synthesis. In contrast, the amount of viral DNA that was integrated into the genomic fraction was greater in mifepristone-treated cells at all time points (FIG. 13C). This increase in the level of integrated viral DNA was the only event in the viral infection cycle that was altered by the presence of mifepristone.

### Example 12

### Mifepristone Increases Gamma-Retroviral Infectivity Through its Effect on the Glucocorticoid Receptor

Mifepristone is a synthetic steroid that can act as an antagonist to both glucocorticoid and progesterone receptors (41). At the molecular level, mifepristone binds to the glucocorticoid or progesterone receptors with high affinity interacting with the receptor at the phenylaminodimethyl group in the 11β-position within a specific region of the receptor binding pocket (42). Mifepristone induces a conformational change within the ligand-binding domain and reduces the receptor's affinity for its natural hormone ligands. Mifepristone's ability to increase viral infectivity of target cells could be due to its effects on either the glucocorticoid receptor or the progesterone receptor.

To determine whether the increase in retroviral infectivity was some specific effect of mifepristone or whether it occurred with other glucocorticoid or progesterone receptor antagonists, rat PMVEC were infected with retrovirus in the presence of mifepristone or another glucocorticoid antagonist, Org 34517, kindly gifted by Schering-Plough. The ic₅₀ of Org 34517 for the glucocorticoid receptor is 17.9 nmol/L (mifepristone's ic₅₀ is 12.2 nmol/L) (43). Org 34517 was used in a range of 0.1-30 μmol/L and the results were compared to rates of infectivity in non-treated or mifepristone-treated cells. Org 34517 significantly increased retroviral infectivity above control but was less potent than mifepristone. A progesterone receptor antagonist {11b-[4-(acetylphenyl)-4′,5′-dihydro-2′-ethyl-5′-methylenespiro[estra-4,9-dien-17b,4′oxazole]-3-one} which has an ic₅₀ of 0.34 nmol/L for the progesterone receptor (graciously provided by RTI International, RTP, NC) (mifepristone's ic₅₀ for the progesterone receptor is 0.054 nmol/L), had no effect on retroviral infection efficiency (FIG. 6A) (44). Adding both the specific glucocorticoid (Org 34517) and the progesterone (7453-102) receptor antagonists at the same time did not change the rate of target cell infectivity (data not shown). This suggests that the improved infection efficiency seen with mifepristone compared to Org 34517 is not due to a synergistic effect of progesterone blockade.

We next examined the effect of mifepristone on retroviral infectivity in cells that lacked either progesterone or glucocorticoid receptors. U87MG cells (a human glioblastoma cell line) lack functional progesterone receptors, but express functional glucocorticoid receptors ¹⁸ whereas T47D cells (a human breast cell line) express progesterone receptors, but do not have functional glucocorticoid receptors (46, 47). Both cell lines were infected with retrovirus in the presence or absence of mifepristone. Seventy-two hours later, cells were analyzed for GFP expression (FIG. 14B). Mifepristone increased retroviral infectivity in U87MG cells, but not in T47D cells. These results, combined with those using selective progesterone and glucocorticoid receptor inhibitors, suggest that mifepristone's ability to increase retroviral infectivity of target cells is likely due to its glucocorticoid receptor antagonist properties.

### Example 13

### Mifepristone Did not Enhance Gamma-Retroviral Integration in T47D Cells

Since mifepristone appeared to enhance target cell infectivity by increasing the rate of retroviral integration into the host genome, (through a glucocorticoid receptor mediated action) we examined whether this effect would be lost in T47D cells. T47D cells were infected with amphotropic MMLV for 1 hr in the presence of 1 μmol/L of mifepristone or vehicle. Virus was then removed and cells were either analyzed immediately or incubated with mifepristone or vehicle for 6 hours, or 1, 3 or 7 days. Similar to previous experiments, the total cellular DNA was isolated and resolved on a 0.8% agarose gel. 4 Kb DNA fragments (the size of non-integrated viral DNA) and genomic DNA (>50 Kb) were cut out and the DNA from each fraction was extracted and analyzed for the presence of viral sequences by qPCR. Similar to our results in endothelial cells (FIG. 13) the level of free (i.e. non-integrated) viral DNA was elevated 6 hours after infection and then gradually declined towards baseline (FIG. 15A). The amount of integrated viral DNA was elevated above baseline 24 hours after infection in both mifepristone and vehicle-treated cells (FIG. 15B), but unlike our results in endothelial cells (FIG. 13) there was no difference in the amount of either free or incorporated viral DNA at any time after infection between mifepristone and vehicle-treated cells. Therefore, it appears that T47D cells, which lack functioning glucocorticoid receptors, are insensitive to mifepristone's ability to increase gamma-retroviral enhanced integration into the host genome. This likely explains the failure of mifepristone to increase viral infection in these cells.

Retroviral vectors are popular and widely used gene delivery vehicles because of their efficiency and precision of integration. Retroviruses can be used to correct genetic diseases through the permanent integration of therapeutic genes into chromosomes of affected cells, for generating transgenic animals and plants, and for basic biomedical research. Determining how retroviruses infect target cells is important not only as a guide towards building better vectors, but to further our understanding of the basic biology of retroviral infection in disease.

MMLV-derived vectors are simple and effective gene delivery vehicles in part because they tend to integrate near transcription initiation sites dramatically increasing expression levels of delivered proteins. The biggest disadvantage of using MMLV-derived vectors is low infection efficiency. While infected cells show a high expression level of delivered protein(s), many cells remain resistant to infection even after several infection cycles. One strategy to increase retroviral infection efficiency is to generate higher viral titers in virus-producing (packaging) cells by maximal activation of enhancers within the retroviral promoter. Since MMLV-derived vectors have one or more response elements within its promoter, glucocorticoids stimulate viral propagation in virus-producing cells that leads to an increase in retroviral infectivity due to an increase in viral titer. We have shown that the glucocorticoid receptor antagonist, mifepristone, blocks this increase in viral titer (28). Our previous work as discussed above in Examples 1-4 demonstrated, however, that while mifepristone blocked the dexamethasone-induced increase in viral titer, it actually increased target cell infectivity. The mechanism underlying that unexpected finding was unclear. As described in Examples 5-13, it appears that mifepristone increased the number of infected target cells by facilitating integration of the viral genome into the host genome. It does not appear to affect any other steps involved with viral infection such as recognition of target cells, virus entry, virus stability, or viral DNA synthesis.

Integration of a retroviral genome into a host chromosome completes the viral replication cycle and makes the infection permanent. The integrated viral DNA can be transcribed, resulting in RNA for translation of viral proteins and formation of viral particles, which can be released to infect other cells. Without viral integration, however, cells eventually degrade the virus and remain uninfected. Mifepristone's ability to increase retroviral infection efficiency appears to be due to its ability to facilitate retroviral integration into the host genome and thus complete the infection. Mifepristone enhanced the efficiency of viral infection in three different gamma-retroviral backbones demonstrating that its effect is not limited to a specific vector. Mifepristone had no effect on the target cell infectivity of the two lentiviral vectors we studied, however. The reason for this is not clear, but may be due to the lack of functional glucocorticoid receptors or other differences in how these lentiviruses infect cells.

The exact mechanism through which mifepristone enhances viral integration in gamma-retroviruses is not clear, but it appears to involve its antagonistic effect on the glucocorticoid receptor. Another anti-glucocorticoid, Org 34517, also increased gamma-retroviral infectivity, whereas an anti-progesterone agent had no effect. Consistent with this observation, mifepristone did not increase gamma-retroviral infection efficiency in cells which lack functional glucocorticoid receptors (T47D cells), but did in those lacking functional progesterone receptors (U87MG cells).

These results suggest that inhibition of the glucocorticoid receptor enhances gamma-retroviral integration into the host genome and indicates that cells have a natural protection again viral infection that may be reduced by glucocorticoid receptor antagonists. As a research tool, mifepristone can be used to enhance gene delivery and provide a way to study different aspects involved in retroviral integration.

### REFERENCES

1. Akahane M, Kuriyama S, Ohgushi H, Akahane T, Kawamura K, Watanabe S, Funakoshi F, Yoshiji H, Ikenaka K, and Takakura Y. Enhancing and suppressing effects of dexamethasone on transgene expression in vitro. Int J Mol Med 10: 107-112, 2002.
2. Barquinero J, Eixarch H, and Perez-Melgosa M. Retroviral vectors: new applications for an old tool. Gene Ther 11 Suppl 1: 53-9, 2004.
3. Beato M, Chalepakis G, Schauer M, and Slater E P. DNA regulatory elements for steroid hormones. J Steroid Biochem 32: 737-747, 1989.
4. Bourgeois S, Pfahl M, and Baulieu E E. DNA binding properties of glucocorticosteroid receptors bound to the steroid antagonist RU-486. Embo J 3: 751-755, 1984.
5. Bruland T, Lavik L A, Dai H Y, and Dalen A. A glucocorticoid response element in the LTR U3 region of Friend murine leukaemia virus variant FIS-2 enhances virus production in vitro and is a major determinant for sex differences in susceptibility to FIS-2 infection in vivo. J Gen Virol 84: 907-916, 2003.
6. Cato A C, Miksicek R, Schutz G, Arnemann J, and Beato M. The hormone regulatory element of mouse mammary tumour virus mediates progesterone induction. Embo J 5: 2237-2240, 1986.
7. Celander D, Hsu B L, and Haseltine W A. Regulatory elements within the murine leukemia virus enhancer regions mediate glucocorticoid responsiveness. J Virol 62: 1314-1322, 1988.
8. Coezy E, Bouhnik J, Clauser E, Pinet F, Philippe M, Menard J, and Corvol P. Effects of glucocorticoids and antiglucocorticoid on angiotensinogen production by hepatoma cells in culture. In Vitro 20: 528-534, 1984.
9. Darbre P, Page M, and King R J. Androgen regulation by the long terminal repeat of mouse mammary tumor virus. Mol Cell Biol 6: 2847-2854, 1986.
10. DeFranco D and Yamamoto K R. Two different factors act separately or together to specify functionally distinct activities at a single transcriptional enhancer. Mol Cell Biol 6: 993-1001, 1986.
11. Duch M, Paludan K, Lovmand J, Pedersen L, Jorgensen P, and Pedersen F S. A correlation between dexamethasone inducibility and basal expression levels of retroviral vector proviruses. Nucleic Acids Res 21: 4777-4782, 1993.
12. Golemis E A, Speck N A, and Hopkins N. Alignment of U3 region sequences of mammalian type C viruses: identification of highly conserved motifs and implications for enhancer design. J Virol 64: 534-542, 1990.
13. Hu W S and Pathak V K. Design of retroviral vectors and helper cells for gene therapy. Pharmacol Rev 52: 493-511, 2000.
14. Jaalouk D E, Eliopoulos N, Couture C, Mader S, and Galipeau J. Glucocorticoid-inducible retrovector for regulated transgene expression in genetically engineered bone marrow stromal cells. Hum Gene Ther 11: 1837-1849, 2000.
15. Kolesnitchenko V and Snart R S. Regulatory elements in the human immunodeficiency virus type 1 long terminal repeat LTR(HIV-1) responsive to steroid hormone stimulation. AIDS Res Hum Retroviruses 8: 1977-1980, 1992.
16. Lundstrom K. Gene therapy applications of viral vectors. Technol Cancer Res Treat 3: 467-477, 2004.
17. Miksicek R, Heber A, Schmid W, Danesch U, Posseckert G, Beato M, and Schutz G. Glucocorticoid responsiveness of the transcriptional enhancer of Moloney murine sarcoma virus. Cell 46: 283-290, 1986.
18. Mitra D, Sikder S K, and Laurence J. Role of glucocorticoid receptor binding sites in the human immunodeficiency virus type 1 long terminal repeat in steroid-mediated suppression of HIV gene expression. Virology 214: 512-521, 1995.
19. Niermann G L and Buehring G C. Hormone regulation of bovine leukemia virus via the long terminal repeat. Virology 239: 249-258, 1997.
20. Otten A D, Sanders M M, and McKnight G S. The MMTV LTR promoter is induced by progesterone and dihydrotestosterone but not by estrogen. Mol Endocrinol 2: 143-147, 1988.
21. Roe T, Reynolds T C, Yu G, and Brown P O. Integration of murine leukemia virus DNA depends on mitosis. Embo J 12: 2099-2108, 1993.
22. Russo F O, Patel P C, Ventura A M, and Pereira C A. HIV-1 long terminal repeat modulation by glucocorticoids in monocytic and lymphocytic cell lines. Virus Res 64: 87-94, 1999.
23. Schafer E A, Venkatachari N J, and Ayyavoo V. Antiviral effects of mifepristone on human immunodeficiency virus type-1 (HIV-1): targeting Vpr and its cellular partner, the glucocorticoid receptor (GR). Antiviral Res 72: 224-232, 2006.
24. Schule R, Muller M, Kaltschmidt C, and Renkawitz R. Many transcription factors interact synergistically with steroid receptors. Science 242: 1418-1420, 1988.
25. Soudeyns H, Geleziunas R, Shyamala G, Hiscott J, and Wainberg M A. Identification of a novel glucocorticoid response element within the genome of the human immunodeficiency virus type 1. Virology 194: 758-768, 1993.
26. Soudeyns H and Wainberg M A. Effects of RU486 on HIV-1 replication. Nat Med 3: 1302-1303, 1997.
27. Warnock J N, Merten O W, and Al-Rubeai M. Cell culture processes for the production of viral vectors for gene therapy purposes. Cytotechnology 50: 141-162, 2006.
28 Solodushko V, Bitko V, Fouty B. Dexamethasone and mifepristone increase retroviral infectivity through different mechanisms. Am J Physiol Lung Cell Mol Physiol 2009; 297: L538-545.
29 D'Souza V, Summers M F. Structural basis for packaging the dimeric genome of Moloney murine leukaemia virus. Nature 2004; 431: 586-590.
30 Hawley R G, Hawley T S, Fong A Z, Quinto C, Collins M, Leonard J P et al. Thrombopoietic potential and serial repopulating ability of murine hematopoietic stem cells constitutively expressing interleukin 11. Proc Natl Acad Sci USA 1996; 93: 10297-10302.
31 Baum C, Eckert H G, Stockschlader M, Just U, Hegewisch-Becker S, Hildinger, M et al. Improved retroviral vectors for hematopoietic stem cell protection and in vivo selection. J Hematother 1996; 5: 323-329.
32 Simons K, Garoff H. The budding mechanisms of enveloped animal viruses. J Gen Virol 1980; 50:1-21.
33 Kim J W, Closs E I, Albritton L M, Cunningham J M. Transport of cationic amino acids by the mouse ecotropic retrovirus receptor. Nature 1991; 352: 725-728.
34 Kozak S L, Siess D C, Kavanaugh M P, Miller A D, Kabat D. The envelope glycoprotein of an amphotropic murine retrovirus binds specifically to the cellular receptor/phosphate transporter of susceptible species. J Virol 1995; 69: 3433-3440.
35 Emi N, Friedmann T, Yee J K. Pseudotype formation of murine leukemia virus with the G protein of vesicular stomatitis virus. J Virol 1991; 65: 1202-1207.
36 Lavillette D, Boson B, Russell S J, Cosset F L. Activation of membrane fusion by murine leukemia viruses is controlled in cis or in trans by interactions between the receptor-binding domain and a conserved disulfide loop of the carboxy terminus of the surface glycoprotein. J Virol 2001; 75: 3685-3695.
37 Lim R Y, Aebi U, Stoffler D. From the trap to the basket: getting to the bottom of the nuclear pore complex. Chromosoma 2006; 115: 15-26.
38 Miller D G, Adam M A, Miller A D. Gene transfer by retrovirus vectors occurs only in cells that are actively replicating at the time of infection. Mol Cell Biol 1990; 10: 4239-4242.
39 Andreadis S T, Brott D, Fuller A O, Palsson B O. Moloney murine leukemia virus-derived retroviral vectors decay intracellularly with a half-life in the range of 5.5 to 7.5 hours. J Virol 1997; 71: 7541-7548.
40 Menendez-Arias L, Berkhout B. Retroviral reverse transcription. Virus Res 2008; 134: 1-3.
41 Spitz I M, Bardin CW. Mifepristone (RU 486)—a modulator of progestin and glucocorticoid action. N Engl J Med 1993; 329: 404-412.
42 Cadepond F, Ulmann A, Baulieu E E. RU486 (mifepristone): mechanisms of action and clinical uses. Annu Rev Med 1997; 48: 129-156.
43 Thomson F, Craighead M W, Watson L, Turnbull Z, McIntosh L, Speake M et al. The compounds Org 34517 and Org 34850 are potent antagonists of the glucocorticoid receptor both in vitro and in vivo. The Journal of the European College of Neuropsychopharmacology 2003; 13: 5229.
44 Jin C, Manikumar G, Kepler J A, Cook C E, Allan G F, Kiddoe M et al. Synthesis and identification of novel 11beta-aryl-4′,5′-dihydrospiro[estra-4,9-diene-17beta,4′-oxazole] analogs with dissociated antiprogesterone activities. Bioorg Med Chem Lett 2007; 17: 5754-5757.
45 Pinski J, Halmos G, Shirahige Y, Wittliff J L, Schally A V. Inhibition of growth of the human malignant glioma cell line (U87MG) by the steroid hormone antagonist RU486. J Clin Endocrinol Metab 1993; 77: 1388-1392.

46 Attardi B J, Zeleznik A, Simhan H, Chiao J P, Mattison D R, Caritis S N. Comparison of progesterone and glucocorticoid receptor binding and stimulation of gene expression by progesterone, 17-alpha hydroxyprogesterone caproate, and related progestins. Am J Obstet Gynecol 2007; 197: 599 e591-597.
47 Kinyamu H K, Fryer C J, Horwitz K B, Archer T K. The mouse mammary tumor virus promoter adopts distinct chromatin structures in human breast cancer cells with and without glucocorticoid receptor. J Biol Chem 2000; 275: 20061-20068.
48 Krishan A. Rapid flow cytofluorometric analysis of mammalian cell cycle by propidium iodide staining. J Cell Biol 1975; 66: 188-193.
49 V. Solodushko and B. Fouty, Mifepristone increases gamma-retroviral infection efficiency by enhancing the integration of virus into the genome of infected cells. Gene Therapy 2010, 1-9.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for enhanced retroviral delivery of a nucleic acid to a target cell in vitro which involves increasing infectivity of a retrovirus carrying a transgene of interest to a target cell comprising culturing the target cell in medium comprising propagated transformed retrovirus and a glucocorticoid receptor antagonist in an amount effective to increase infectivity of the retrovirus to the target cell.

2. The method of claim 1 wherein the glucocorticoid receptor antagonist enhances integration of the retrovirus into the target cell.

3. The method of claim 1 wherein the glucocorticoid receptor antagonist is mifepristone (RU-486), or an analog thereof.

4. The method of claim 1 wherein the propagated retrovirus is obtained from a frozen stock of retroviral supernatant.

5. The method of claim 1 wherein the glucocorticoid receptor antagonist is added to the target cell cultures before, at about the same time, or after the addition of the retrovirus.

6. The method of claim 1 wherein the medium comprises another cell infectivity enhancing agent.

7. A method for increasing retroviral infectivity of target cells comprising:
a) culturing packaging cells transfected with retroviral vector containing a transgene of interest in medium containing a glucocorticoid receptor agonist present in the medium in an amount effective to increase titer of propagated retrovirus; and
b) subsequently culturing a target cell in medium comprising the propagated retrovirus from a) and a glucocorticoid receptor antagonist present in the medium in an amount effective to increase target cell sensitivity to infection by the propagated retrovirus.

8. The method of claim 7 wherein the glucocorticoid receptor agonist is dexamethasone, or an analog thereof.

9. The method of claim 7 wherein the glucocorticoid receptor antagonist is mifepristone or an analog thereof.

10. The method of claim 7 wherein the target cells are cultured with retrovirus-containing culture medium obtained from packaging cell cultures or with retrovirus that has been obtained from packaging cell culture medium.

11. The method of claim 7 wherein the retrovirus is a Gammaretrovirus.

12. The method of claim 1 wherein the retrovirus is a Gammaretrovirus.

13. A kit comprising a glucocorticoid receptor agonist and a glucocorticoid receptor antagonist in amounts sufficient to increase retroviral titer and target cell sensitivity to retroviral infection, respectively.

14. The kit of claim 13 further comprising one or more additional retroviral infectivity enhancers.
